(19) United States
(12) Patent Application Publication
(10) Pub. No.: US 2024/0189266 A1
(43) Pub. Date: Jun. 13, 2024

(54) Lactoyl Amino Acids for the Treatment of Metabolic Disease

(71) Applicants: The Board of Trustees of the Leland Stanford Junior University, Stanford, CA (US); Baylor College of Medicine, Houston, TX (US)

(72) Inventors: Jonathan Long, Stanford, CA (US); Veronica Li, Stanford, CA (US); Steven Banik, Stanford, CA (US); Yong Xu, Houston, TX (US); Yang He, Houston, TX (US)

(21) Appl. No.: 18/286,516

(22) PCT Filed: May 2, 2022
(86) PCT No.: PCT/US2022/027261

§ 371 Date: Oct. 11, 2023

Related U.S. Application Data
(60) Provisional application No. 63/183,868, filed on May 4, 2021.

(51) Int. Cl. A61K 31/198; A61K 45/06; A61P 3/04

(52) U.S. Cl. CPC A61K 31/198; A61K 45/06; A61P 3/04

(57) ABSTRACT

Methods of treating a metabolic disorder in a subject are provided. Aspects of the method include administering an effective amount of an N-lactoyl-amino acid to the subject. Also provided are pharmaceutical formulations including an amount of an N-lactoyl-amino acid effective to treat a metabolic disorder. Any suitable N-lactoyl-amino acid or combination of N-lactoyl-amino acids may be administered in the subject methods.

### CROSS-REFERENCE TO RELATED APPLICATION

Pursuant to 35 U.S.C. § 119 (e), this application claims priority to the filing date of U.S. Provisional Patent Application Ser. No. 63/183,868 filed May 4, 2021, the disclosure of which application is incorporated herein by reference in its entirety.

### INTRODUCTION

Metabolic disorders generally refer to a broad array of disorders characterized by defects that interfere with the body's metabolism, the chemical processes by which a body transforms proteins, carbohydrates and fats into energy. Metabolic disorders may include disorders resulting from altered glucose metabolism. Examples of metabolic disorders include obesity, metabolic syndrome, impaired glucose tolerance, and dyslipidemias. A metabolic disorder can also result from a diseased or dysfunctional organ. Diabetes is an example of a metabolic disorder resulting from a diseased and/or dysfunctional organ, the pancreas.

Obesity, which is defined in general terms as an excess of body fat relative to lean body mass, is a serious contributor to increased morbidity and mortality. Obesity, which is most commonly caused by excessive food intake coupled with limited energy expenditure and/or lack of physical exercise, often accompanies various glucose metabolism disorders. Obesity increases the likelihood of an individual developing various diseases, such as diabetes mellitus, hypertension, atherosclerosis, coronary artery disease, gout, rheumatism and arthritis. Obesity is often associated with psychological and medical morbidities, the latter of which includes increased joint problems, vascular diseases such as coronary artery disease, hypertension, stroke, and peripheral vascular disease. Obesity also causes metabolic abnormalities such as insulin resistance and Type II diabetes (non-insulin-dependent diabetes mellitus (NIDDM)), hyperlipidemia, and endothelial dysfunction.

### SUMMARY

Methods of treating a metabolic disorder in a subject are provided. Aspects of the method include administering an effective amount of an N-lactoyl-amino acid to the subject. Also provided are pharmaceutical formulations including an amount of an N-lactoyl-amino acid effective to treat a metabolic disorder.

### BRIEF DESCRIPTION OF THE FIGURES

FIG. 1 A-D shows Lac-Phe is robustly induced in blood plasma after a single bout of mouse treadmill running. (A) Schematic of the speed and incline protocol for treadmill running in mice. (B) T-stat values of all blood plasma peaks detected by untargeted metabolomics in post-run versus sedentary mice. (C) Extracted ion chromatogram of the m/z=236.0928 mass in the post-exercise (red traces) or sedentary (blue traces) blood plasma. (D) Tandem MS fragmentation (left) and structural assignment (right) of an authentic Lac-Phe standard (top) and endogenous m/z=236.0928 mass (bottom). For B and C, N=5/group.

FIG. 2 A-I shows Lac-Phe suppresses food intake and obesity and improves glucose homeostasis. (A-B) Cumulative food intake (A) and ambulatory activity (B) of 22-week male DIO mice following injection of either vehicle (blue) or Lac-Phe (red, 50 mg/kg, intraperitoneal [IP]). (C-D) Cumulative food intake (C) and change in body weight (D) of 22-week male DIO mice treated daily with vehicle (blue) or Lac-Phe (red, 50 mg/kg/day, intraperitoneal [IP]). (E) Glucose tolerance test (1 g/kg glucose) of vehicle- or Lac-Phe treated mice. GTT was performed one day after the last Lac-Phe dose on day 10 following a 6 h fast. (F-G) Tissue weights (F) and representative images of adipose tissues (G) from mice after 10 days of vehicle or Lac-Phe treatment. (H) Average daily food intake (left) and change in body weight (right) of 15-week male DIO mice after 5-day treatment with vehicle (black), Lac-Phe (red, 50 mg/kg/day, IP) or vehicle-treated pair-fed mice (blue). (I) Average daily food intake (left) and change in body weight (right) of 13-week male DIO mice after 7-day treatment with vehicle (black), lactate (blue, 50 mg/kg/day, IP), phenylalanine (grey, 50 mg/kg/day, IP), or Lac-Phe (red, 50 mg/kg/day, IP). For (A-B), N=6/group; for (C-H), N=8-10/group; for (I), N=5/group. Data are shown as mean±SEM; *p<0.05, **p<0.01, ***p<0.001.

FIG. 3 A-H shows CNDP2- and lactate-dependent biosynthesis and secretion of Lac-Phe from macrophages in vitro. (A) Lac-Phe levels in conditioned media and cell lysate of RAW264.7 cells. (B) Anti-CNDP2 or anti-beta tubulin Western blotting of cell lysates from WT or CNDP2-KO RAW264.7 cells. (C) Lac-Phe levels in conditioned media and cell lysate of WT and CNDP2-KO RAW264.7 cells. (D) Anti-CNDP2 or anti-beta tubulin Western blotting of cell lysates from primary peritoneal macrophages isolated from WT or CNDP2-KO mice. (E) Lac-Phe levels in conditioned media and lysate of WT and CNDP2 KO primary peritoneal macrophages. (F, G) Lac-Phe levels in conditioned media and lysate of WT RAW264.7 (F) and WT peritoneal macrophages (G) following treatment with lactate (25 mM). (H) Schematic of Lac-Phe synthesis in vitro. For (A), (C), and (E-G), n=3-5/group. Data are shown as means±SEM, **p<0.01.

FIG. 4 A-F shows genetic ablation of Lac-Phe biosynthesis results in increased food intake and obesity. (A) Anti-CNDP2 Western blotting of tissue lysates in WT mice. (B) Relative abundance of Lac-Phe in plasma of male WT (blue) and CNDP2 KO (red) mice under sedentary and exercised conditions. (C-F) Cumulative daily food intake (C), body weight (D), tissue weights (E), and representative images of adipose tissues (F) of WT (blue) and CNDP2-KO (red) mice under an obesigenic diet/exercise training regimen in which mice were fed high fat diet (60% kcal from fat) and exercised by treadmill running 5 days/week (see Methods). Tissue weights and images were taken on day 41. For (B), N=6/group. For (C-F), N=8-9/group. Data are shown as mean±SEM, *p<0.05, **p<0.01, ***p<0.001.

FIG. 5 A-J shows Lac-Phe acts directly on AgRP+ and POMC+ neurons in culture. (A) Representative action potential firing traces of AgRP+ neurons after treatment with vehicle (left) or Lac-Phe (50 μM, right). (B,C) Effects of the indicated concentration of Lac-Phe on action potential firing frequency (B) and hyperpolarization (C) of AgRP+ neurons. (D) Representative electrophysiology recording of AgRP+ neurons after treatment with Lac-Phe (50 μM) in the presence of an inhibitor cocktail (tetrodotoxin: 1 μM, bicuculline: 50 μM, DNQX: 20 μM, and D-AP5: 50 μM). (E) Response ratio of AgRP+ neurons after treatment with various concentrations of Lac-Phe alone or in the presence of an inhibitor cocktail (F) Representative action potential firing traces of POMC+ neurons after treatment with vehicle (left) or Lac-Phe (50 μM, right). (G,H) Effects of the indicated concentrations of Lac-Phe on action potential firing frequency (G) and hyperpolarization (H) of POMC+ neurons. (1) Representative electrophysiology recording of POMC+ neurons after treatment with Lac-Phe (50 μM) in the presence of an inhibitor cocktail. (J) Response ratio of POMC+ neurons after treatment with various concentrations of Lac-Phe alone or in the presence of an inhibitor cocktail. For (A-J), N=8-15 neurons/group. Data shown as mean±SEM, **p<0.01, ***p<0.001.

FIG. 6 A-F shows robust and sustained elevation of Lac-Phe following human exercise. (A) Schematic of human acute treadmill exercise study design, N=36. (B) False discovery rate of exercise-regulated metabolites (dark blue), lipids (grey), or proteins (light blue) from blood plasma in response to acute treadmill running. A previously unassigned metabolite with a chemical formula matching that of Lac-Phe is shown in red. (C) Time course of Lac-Phe and lactate in blood in humans following exercised or kept sedentary. (D) Schematic of human crossover acute exercise study design, N=8. (E) Time course of Lac-Phe levels pre and post-exercise following the sprint (red), resistance (blue), and endurance (light blue) trial. (F) Correlation of plasma Lac-Phe and lactate levels immediately pre- and post-exercise across the three exercise modalities. Data are shown as mean±SEM, **p<0.01, ***p<0.001, ****p<0.0001.

FIG. 7 A-B shows positive metabolite controls and identification of m/z=236 peak. (A) Fold-change of the indicated metabolites in exercise versus sedentary conditions. N=5/group, data are shown as means±SEM, **p<0.01, ***p<0.001. (B) Co-elution of endogenous m/z=236.0928 mass with an authentic Lac Phe standard.

FIG. 8 A-C shows additional metabolic parameters of obese mice acutely treated with Lac-Phe. (A-C) 12 hr oxygen consumption VO2 (A), carbon dioxide production VCO2 (B), and respiratory exchange ratio RER (C) of 22-week old DIO mice following a single injection of vehicle or Lac-Phe (50 mg/kg, IP). N=6/group, data are shown as means±SEM.

FIG. 9 A-C shows characterization of Lac-Phe production in vitro. (A) Lac-Phe levels in conditioned media from a panel of cell lines after overnight incubation. (B) Percent abundance of the indicated metabolite in cell lysates or conditioned media of RAW264.7 cells after overnight incubation. (C) Diagram of the CNDP2-catalyzed Lac-Phe biosynthesis reaction. For (A,B), data are shown as mean±SEM, *p<0.05.

FIG. 10 A-D shows the effect of Lac-Phe on serotonin and SF1 neurons in brain slices. (A,B) Representative action potential firing trace of Lac-Phe (50 μM) treatment of 5-HT+neurons (A) and the response ratio (B). (C,D) Representative action potential firing trace of Lac-Phe (50 μM) treatment of SF1+ neurons (C) and the response ratio (D). For (C-D), N=14-15 neurons/group.

FIG. 11 A-D shows additional characterization of plasma Lac-Phe levels in humans. (A,B) Tandem MS fragmentation (A) and co-elution (B) of an authentic Lac-Phe standard (blue) and the endogenous m/z=236.0928 mass from human plasma (red). (C) Time course of Lac-Phe (red), lactate (blue), and phenylalanine (light blue) levels in blood before and after a single acute bout of treadmill running from the human acute treadmill exercise study (cohort 1, N=36). (D) Time course of lactate levels before and after sprint (red), resistance (blue), and endurance (light blue) exercise from the human crossover acute exercise study (cohort 2, N=8). For (C-D), data are shown as mean±SEM, **p<0.01, ***p<0.001.

FIG. 12 A-E shows additional characterization of the mouse exercise plasma metabolome. (A) Fold change of metabolites detected in our untargeted metabolomics analysis that are known to be increased with physical activity. Basal and exercised conditions are indicated in blue and red respectively. (B) Co-elution of an authentic Lac-Phe standard (blue) and the endogenous m/z=236.0928 mass from mouse exercise plasma (red). (C) Absolution quantification of Lac-Phe in mouse plasma under sedentary (blue) and exercised (red) conditions. (D) Total ion count of the 20 N-lactoyl amino acid conjugates in the plasma of mice under basal (blue) and exercised (red) conditions. (E) Time course of Lac-Phe in the plasma of mice under basal conditions, immediately post-exercise, and 30 minutes and 120 minutes post-exercise. For (A, C-E), studies were performed with N=3-5 per group and data are shown as mean±SEM, *p<0.05, **p<0.01, ***p<0.001, ****p<0.0001.

FIG. 13 A-J shows Lac-Phe acts directly on AgRP+ and POMC+ neurons in culture and contains similar information to FIG. 5 (above). (A) Representative action potential firing traces of AgRP+ neurons after treatment with vehicle (left) or Lac-Phe (50 μM, middle) or Lac-Phe (50 μM) in the presence of an inhibitor cocktail (tetrodotoxin: 1 μM, bicuculline: 50 μM, DNQX: 20 μM, and D-AP5: 50 μM, right). (B) Response ratio of AgRP+ neurons after treatment with Lac-Phe alone (left, n=12 neurons) or in the presence of the inhibitor cocktail (right, n=9 neurons). (C) Quantification of the firing frequency of AgRP+ neurons before and after Lac-Phe treatment. (D-E) Quantification of the resting membrane potential of AgRP+ neurons before and after Lac-Phe treatment alone (D) or in the presence of an inhibitor cocktail (E). (F-J) Effects of Lac-Phe on POMC+ neurons. (F) Representative action potential firing traces of POMC+ neurons after treatment with vehicle (left), Lac-Phe (50 μM, middle), or Lac-Phe in the presence of an inhibitor cocktail (50 μM, right). (G) Response ratio of POMC+ neurons after treatment with Lac-Phe alone (left, n=16 neurons) or in the presence of an inhibitor cocktail (right, n=15 neurons). (H) Quantification of the firing frequency of POMC+ neurons before and after Lac-Phe treatment. (I-J) Quantification of the resting membrane potential of POMC+ neurons before and after Lac-Phe treatment alone (I) or in the presence of an inhibitor cocktail (J). For (A-J), N=8-15 neurons/group. Data shown as mean, **p<0.01, ***p<0.001.

FIG. 14 A-E shows human exercise induces sustained elevations of Lac-Phe and negative correlations with BMI and fat mass. (A) Schematic of human acute treadmill exercise study design, N=36. (B) False discovery rate of exercise-regulated metabolites (dark blue), lipids (grey), or proteins (light blue) from blood plasma in response to acute treadmill running. A previously unassigned metabolite with a chemical formula matching that of Lac-Phe is shown in red. (C) Time course of Lac-Phe (red), lactate (blue), and phenylalanine (light blue) in the plasma of subjects pre and post-exercise. (D) Associations of circulating Lac-Phe levels 30 minutes post-exercise with demographics and physiological markers. (E) Associations of circulating Lac-Phe levels 30 minutes post-exercise with BMI (left) and estimated fat mass (right).

FIG. 15 shows Lac-Phe is also present in humans and changes in circulating levels with exercise and food intake. (Top) Schematic of study design with indications of when exercise and food consumption occurred and when blood was sampled, N=36. (Bottom) Time course of Lac-Phe (red), lactate (blue), and phenylalanine (green) in subjects pre and post-exercise (left) and throughout the experiment including post-food intake (right).

FIG. 16 shows peak exercised-induced Lac-Phe is negatively associated with body mass index and fat mass. Associations of circulating Lac-Phe levels 30 minutes post-exercise with BMI (left), estimated fat mass (middle), and percent fat mass (right).

FIG. 17 shows administration of Lac-Phe to diet-induced obese mice suppresses appetite and body weight and improves glucose homeostasis. (Top) Body weight (left) and change in body weight (right) of 18 week male DIO mice after 9-day treatment with vehicle (blue) or Lac-Phe (red, 50 mg/kg/day, IP). (Bottom) Glucose tolerance test (1 g/kg glucose, left) of vehicle or Lac-Phe treated mice. GTT was performed following a 6 h fast. Cumulative food intake (right) of mice following injection of either vehicle (blue) or Lac-Phe (red, 50 mg/kg, intraperitoneal, IP). N=10/group. Data are shown as mean±SEM; *p<0.05, **p<0.01.

FIG. 18 shows the anti-obesity effect of Lac-Phe requires the intact amide conjugate. Change in body weight of 13-week male DIO mice after 7-day treatment with vehicle (black), lactate (blue, 50 mg/kg/day, IP), phenylalanine (green, 50 mg/kg/day, IP), or Lac-Phe (red, 50 mg/kg/day, IP). N=5/group. Data are shown as mean±SEM; *p<0.05.

FIG. 19 A-C shows Lac-Phe is a metabolite secreted from macrophages in a lactate and CNDP2-dependent manner. (A) Lac-Phe levels in conditioned media from a panel of cell lines after overnight incubation. (B) Lac-Phe levels in conditioned media and cell lysate of RAW264.7 cells. (C) Anti-CNDP2 or anti-beta tubulin Western blotting of cell lysates from WT or CNDP2-KO RAW264.7 cells (left). Lac-Phe levels in conditioned media and cell lysate of WT and CNDP2-KO RAW264.7 cells (middle). Lac-Phe levels in conditioned media and lysate of WT RAW264.7 cells following treatment with lactate (25 mM). For (A-C) N=3-5/group. Data are shown as means±SEM, *p<0.05, **p<0.01.

FIG. 20 C-F shows mice without Lac-Phe exhibit increased food intake and obesity. (C-F) Body weight, (C) cumulative food intake (D), images (E), and tissue weights (F) of WT (blue) and CNDP2-KO (red) mice under an obesigenic diet/exercise training regimen in which mice were fed high fat diet (60% kcal from fat) and exercised by treadmill running 5 days/week (see Methods). For (C-F) N=8-9/group. Data are shown as mean±SEM, *p<0.05, **p<0.01, ***p<0.001.

FIG. 21 shows CNDP2-KO mice have abolished Lac-Phe levels in blood. Relative abundance of Lac-Phe in plasma of male WT (blue) and CNDP2 KO (red) mice under sedentary and exercised conditions. N=6/group data is shown as mean±SEM, *p<0.05, ***p<0.001.

FIG. 22 A-B Change in body weight (a) and daily food intake (b) of 14-week DIO mice following injection of either vehicle (purple), Lac-Phe (blue), Lac-Leu (gray), Lac-Ile (light blue), Lac-Val (green), or Lac-Met (teal). Treatments were at 50 mg/kg/day, IP, n=4.

### DETAILED DESCRIPTION

Methods of treating a metabolic disorder in a subject are provided. Aspects of the method include administering an effective amount of an N-lactoyl-amino acid to the subject. Also provided are pharmaceutical formulations including an amount of an N-lactoyl-amino acid effective to treat a metabolic disorder.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

In further describing various aspects of the invention, the methods are reviewed first in greater detail, followed by a review of pharmaceutical formulations that find use in embodiments of the methods.

### Methods

As summarized, above, methods of treating a metabolic disorder in a subject are provided. The methods may include administering an effective amount of an N-lactoyl-amino acid to the subject. As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g., obesity. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease. A metabolic disorder may be “treated” if at least one symptom of the metabolic disorder is expected to be or is alleviated, terminated, slowed, or prevented. A metabolic disorder may be also “treated” if recurrence or progression of the metabolic disorder is reduced, slowed, delayed, or prevented.

Any suitable N-lactoyl-amino acid or combination of N-lactoyl-amino acids may be administered in the subject methods. As used herein in its conventional sense, “N-lactoyl-amino acids” or “lac-amino acids” refer to compounds, e.g., metabolites, that are formed from lactate and amino acids, e.g., by the action of protease cytosolic nonspecific dipeptidase 2 (CNDP2). The N-lactoyl-amino acid may be one that is produced by the body or one that is synthetically produced. The amino acid in the N-lactoyl-amino acid(s) that are administered may be any suitable amino acid including, e.g., phenylalanine, isoleucine, valine, glycine, methionine, glutamic acid, tryptophan, alanine, asparagine, glutamine, histidine, serine, proline, threonine, cysteine, lysine, arginine, tyrosine, aspartic acid, leucine, etc. In some instances, the N-lactoyl-amino acid(s) administered in the subject methods are those that are present (e.g., detectable) in the body during or after physical activity. In some instances, the N-lactoyl-amino acid(s) administered in the subject methods are those where the levels of the N-lactoyl-amino acid(s) are elevated or decreased in the body, e.g., blood plasma, during or after physical activity. In some instances, the subject methods include administering an analog of any of the N-lactoyl-amino acids described herein. In some instances, the N-lactoyl-amino acid is N-lactoyl-phenylalanine (e.g., Lac-Phe). In some instances, the N-lactoyl-amino acid is N-lactoyl-leucine (e.g., Lac-Leu). In some instances, the N-lactoyl-amino acid is N-lactoyl-isoleucine (e.g., Lac-Ile). In some instances, the N-lactoyl-amino acid is N-lactoyl-valine (e.g., Lac-Val). In some instances, a plurality of N-lactoyl-amino acids are administered including, e.g., two or more, three or more, four or more, five or more, six or more, seven or more, eight or more, nine or more, or ten or more N-lactoyl-amino acids. In some instances, the N-lactoyl-amino acid is not N-lactoyl-methionine (e.g., Lac-Met).

Any suitable amount of the N-lactoyl-amino acid may be administered. In some instances, an amount effective to treat a metabolic disease or associated condition, e.g., an effective amount, is administered. In some instances, an amount of each N-lactoyl-amino acid in a plurality of N-lactoyl-amino acids effective to treat a metabolic disease or associated condition is administered. For example, where a plurality of N-lactoyl-amino acids are administered, the effective amount for each of the N-lactoyl-amino acids may be administered. In some instances, the effective amount includes an amount of the N-lactoyl-amino acid that when administered produces a plasma concentration of the N-lactoyl-amino acid comparable (e.g., equivalent) to that observed in the subject during or after physical activity. For example, the effective amount may be equivalent to the amount of the N-lactoyl-amino acid present in the body, e.g., blood plasma, of the subject during or after physical activity. In some instances, the effective amount includes an amount of the N-lactoyl-amino acid that when administered produces a plasma concentration of the N-lactoyl-amino acid comparable (e.g., equivalent) to that observed in the subject during or after a recovery period after physical activity (e.g., a period of time after physical activity that ranges from 1 minute to 5 hours including, e.g., from 1 minute to 4 hours, from 1 minute to 3 hours, from 1 minute to 2 hours, from 1 minute to 1 hour, from 1 minute to 30 minutes, or from 1 minute to 15 minutes). For example, the effective amount may be equivalent to the amount present in the body, e.g., plasma, during or after a recovery period after physical activity. Physical activity may include any amount of physical activity over a period of time. In some instances, physical activity includes an amount of physical activity performed over a period of time ranging from 1 minute to 2 hours including, e.g., from 1 minute to 1 hour, or from 1 minute to 30 minutes. Physical activity may include, e.g., any activity that raises the heart rate above a resting heart rate, physical movement, exercise, any activity performed to maintain or achieve physical fitness, etc. In some instances, the effective amount includes a single unit dose of the N-lactoyl-amino acid. In some instances, the effective amount includes one or more unit doses of the N-lactoyl-amino acid including, e.g., two or more doses, three or more doses, four or more doses, etc. In some instances, a single dose is administered. In some instances, multiple doses, e.g., two or more, three or more, etc., are administered. The effective amount may range from 1 mg/kg to 500 mg/kg including, e.g., from 1 mg/kg to 400 mg/kg, from 1 mg/kg to 300 mg/kg, from 1 mg/kg to 200 mg/kg, from 1 mg/kg to 100 mg/kg.

In some instances, the methods include administering an amount of the N-lactoyl-amino acid(s) effective to induce a physical activity associated outcome in a subject (e.g., effective to cause the subject to experience a physical activity associated outcome). By “physical activity associated outcome” is meant an outcome, change, or effect (e.g., biological, physical, and/or chemical) equivalent to that induced in the subject by physical activity (e.g., equivalent in magnitude and longevity). Physical activity associated outcomes of interest include, but are not limited to, weight loss, prevention or treatment of metabolic disorders and associated conditions, improved glucose homeostasis, improved thinking or cognition, mood improvement, reduction in the severity of mood disorders (e.g., anxiety and depression), prevention of or slowing the progress of neurodegenerative diseases (e.g., dementia), improvement in sleep, lower risk of cancer, among others. For example, the methods may include administering an amount of the N-lactoyl-amino acid effective to induce a reduction in body weight in the subject equivalent to what would be induced by physical activity. In another example, the methods may include administering an amount of the N-lactoyl-amino acid effective to induce an improvement in glucose homeostasis in the subject equivalent to what would be induced by physical activity. In another example, the methods may include administering an amount of the N-lactoyl-amino acid effective to induce a mood improvement in the subject equivalent to what would be induced by physical activity. In yet another example, the methods may include administering an amount of the N-lactoyl-amino acid effective to induce a reduction in the severity of a mood disorder in the subject equivalent to what would be induced by physical activity. In yet another example, the methods may include administering an amount of the N-lactoyl-amino acid effective to induce a reduction in the severity of a neurodegenerative disorder (e.g., prevention of the development of the disorder or slowing of the progression of the disorder) in the subject equivalent to what would be induced by physical activity. In some embodiments, the methods include administering an amount of the N-lactoyl-amino acid effective to treat a nervous system disorder and/or associated conditions. The nervous system disorder may include, e.g., mood or psychiatric disorders (e.g., anxiety, depression, bipolar disorder, seasonal affective disorder, etc.) or a neurodegenerative disease (e.g., dementia, Alzheimer's disease, Parkinson's disease, etc.). The effective amount may be any of the amounts described herein.

In some instances, the N-lactoyl-amino acid(s) may be administered according to a dosing schedule. In some instances, the effective amount is administered once to the subject. In some instances, the effective amount is administered once a day to the subject. In some instances, the effective amount is administered multiple times a day to the subject. In some instances, the effective amount is administered once a day over a period of time ranging from 1 day to 60 days, e.g., from 1 day to 10 days, from 1 day to 7 days, from 1 day to 5 days, or from 1 day to 3 days. In some instances, the effective amount is administered from 1 time to 5 times per day including, e.g., 1 time to 3 times per day, 2 times to 5 times per day, or 3 times to 5 times per day, over a period of time ranging from 1 day to 14 days, e.g., from 1 day to 10 days, from 1 day to 7 days, from 1 day to 5 days, or from 1 day to 3 days.

In certain embodiments, the methods include administering the N-lactoyl-amino acid, e.g., N-lactoyl-phenylalanine, or combination of N-lactoyl-amino acids in combination with one or more therapies for treating a metabolic disorder and/or associated condition, e.g., obesity. In some instances, the methods include administering the N-lactoyl-amino acid in combination with an active agent (or a combination of one or more active agents) for treating the metabolic disorder. In some instances, the methods include administering the N-lactoyl-amino acid in combination with an active agent for treating obesity. Active agents of interest include, but are not limited to, orlistat, lorcaserin, phentermine-topiramate, naltrexone-bupropion, liraglutide, phentermine, benzphetamine, diethylpropion, phendimetrazine, among others. In some instances, the therapies that may be used in combination with the N-lactoyl-amino acid to treat the metabolic disorder (e.g., obesity) include any one of or a combination of any of the following therapies: physical activity, a dietary plan (e.g., a low fat diet, low calorie diet, intermittent fasting, etc.), use of a weight loss device, and surgical intervention (e.g., bariatric surgery). Weight loss devices may include, e.g., an electrical stimulation system (e.g., a device that blocks nerve activity between the stomach and brain), a gastric balloon system (e.g., one or more balloons placed in the stomach), and/or a gastric emptying system (e.g., a pump and tube to drain food from the stomach after a meal).

In some instances, the one or more therapies include a small molecule agent. Naturally occurring or synthetic small molecule compounds of interest include numerous chemical classes, such as organic molecules, e.g., small organic compounds having a molecular weight of more than 50 and less than about 2,500 Daltons. Candidate agents comprise functional groups for structural interaction with proteins, particularly hydrogen bonding, and typically include at least an amine, carbonyl, hydroxyl or carboxyl group, preferably at least two of the functional chemical groups. The candidate agents may include cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Such molecules may be identified, among other ways, by employing the screening protocols.

In some instances, the one or more therapies include a protein or a fragment thereof or a protein complex. In some embodiments, the one or more therapies include an antibody binding agent or derivative thereof. The term “antibody binding agent” as used herein includes polyclonal or monoclonal antibodies or fragments that are sufficient to bind to an analyte of interest. The antibody fragments can be, for example, monomeric Fab fragments, monomeric Fab′ fragments, or dimeric F(ab)′2 fragments. Also within the scope of the term “antibody binding agent” are molecules produced by antibody engineering, such as single-chain antibody molecules (scFv) or humanized or chimeric antibodies produced from monoclonal antibodies by replacement of the constant regions of the heavy and light chains to produce chimeric antibodies or replacement of both the constant regions and the framework portions of the variable regions to produce humanized antibodies. In some cases, the one or more therapies include an enzyme or enzyme complex. In some cases, the one or more therapies includes a phosphorylating enzyme, e.g., a kinase. In some cases, the one or more therapies includes a complex including a guide RNA and a CRISPR effector protein, e.g., Cas9, used for targeted cleavage of a nucleic acid.

In some embodiments, the one or more therapies includes a nucleic acid. The nucleic acids may include DNA or RNA molecules. In certain embodiments, the nucleic acids modulate, e.g., inhibit or reduce, the activity of a gene or protein, e.g., by reducing or downregulating the expression of the gene. The nucleic acid may be a single stranded or double-stranded and may include modified or unmodified nucleotides or non-nucleotides or various mixtures and combinations thereof. In some cases, the one or more therapies includes intracellular gene silencing molecules by way of RNA splicing and molecules that provide an antisense oligonucleotide effect or an RNA interference (RNAi) effect useful for inhibiting gene function. In some cases, gene silencing molecules, such as, e.g., antisense RNA, short temporary RNA (stRNA), double-stranded RNA (dsRNA), small interfering RNA (siRNA), short hairpin RNA (shRNA), microRNA (miRNA), tiny non-coding RNA (tncRNA), snRNA, snoRNA, and other RNAi-like small RNA constructs, may be used to target a protein-coding as well as non-protein-coding genes. In some case, the nucleic acids include aptamers (e.g., spiegelmers). In some cases, the nucleic acids include antisense compounds. In some cases, the nucleic acids include molecules which may be utilized in RNA interference (RNAi) such as double stranded RNA including small interfering RNA (siRNA), locked nucleic acid (LNA) inhibitors, peptide nucleic acid (PNA) inhibitors, etc.

The N-lactoyl-amino acid(s) may be administered by any suitable means. As used herein, the term “administering” includes in vivo administration as well as direct administration to tissues ex vivo. Generally, administration is, for example, oral, buccal, parenteral (e.g., intravenous, intraarterial, subcutaneous), intraperitoneal (i.e., into the body cavity), topically, e.g., by inhalation or aeration (i.e., through the mouth or nose), or rectally systemic (i.e., affecting the entire body). A composition may be administered in dosage unit formulations containing conventional non-toxic pharmaceutically acceptable carriers, adjuvants, and vehicles as desired. The term “topically” may include injection, insertion, implantation, topical application, or parenteral application.

In some embodiments, the N-lactoyl-amino acid is administered in a pharmaceutical formulation or as a pharmaceutically acceptable composition in which one or more N-lactoyl-amino acids may be mixed with one or more carriers, thickeners, diluents, buffers, preservatives, surface active agents, excipients and the like. Pharmaceutical compositions may also include one or more additional active ingredients such as antimicrobial agents, anti-inflammatory agents, anesthetics, and the like in addition to the one or more N-lactoyl-amino acids. In some cases, the N-lactoyl-amino acid composition includes, e.g., a derivative or analog of an N-lactoyl-amino acid. “Derivatives” include pharmaceutically acceptable salts and chemically modified agents. “Analogs” include a chemical compound that is structurally similar to another but differs slightly in composition (as in the replacement of one atom by an atom of a different element or in the presence of a particular functional group, or the replacement of one functional group by another functional group). Thus, an analog may be a compound that is similar or comparable in function and appearance, but not in structure or origin to the reference compound. The pharmaceutical compositions may be administered by any route commonly used to administer pharmaceutical compositions. For example, administration may be done topically (including opthalmically, vaginally, rectally, intranasally), orally, by inhalation, or parenterally, for example by intravenous drip or subcutaneous, intraperitoneal or intramuscular injection. The pharmaceutical composition including the N-lactoyl-amino acid may be stored at any suitable temperature. In some cases, the N-lactoyl-amino acid composition is stored at temperatures ranging from 1° C. to 30° C., from 2° C. to 27° C., or from 5° C. to 25° C. The N-lactoyl-amino acid composition may be stored in any suitable container, as described in detail below.

The metabolic disorder treated by the subject methods may vary. By “metabolic disorder,” “metabolic condition,” “metabolic disease,” “metabolic disease-associated condition,” or “metabolic disorder-associated condition” is meant a disorder or condition relating to abnormality of metabolism. In some instances, a “metabolic disorder” refers to any disorder associated with or aggravated by impaired or altered glucose regulation or glycemic control, such as, for example, insulin resistance. Such disorders include, but are not limited to, diabetes, hyperglycemia, obesity, etc. Metabolic disorders and conditions associated with metabolic disorders that can be treated according to the methods described herein include but are not limited to overweight, obesity, hyperphagia, diabetes (inclusive of type 1 diabetes and type 2 diabetes), type 2 diabetes, impaired glucose tolerance, insulin resistance, hyperinsulinemia, dyslipidemia, hypertension, metabolic syndrome. The disorder treated by the subject methods may also be obesity and metabolic syndrome associated disorders, such as but not limited to, meningioma, adenocarcinoma, multiple myeloma, kidney cancer, endometrium cancer, ovarian cancer, colorectal cancer, pancreatic cancer, stomach cancer, gallbladder cancer, liver cancer, breast cancer, thyroid cancer, and any other obesity associated cancers. Including osteoarthritis, stroke, gallbladder disease, chronic kidney disease, and coronary artery disease. Including mental disorders such as clinical depression and anxiety, bipolar disorder, panic disorder, and agoraphobia. Metabolic disorders that can be treated according to the methods described herein can be included in embodiments individually or in any combination.

In some instances, the metabolic disorder or metabolic disorder-associated condition is obesity. The term “obesity” refers to a condition characterized by an excess of body fat. The operational definition of obesity may be based on the Body Mass Index (BMI), which is calculated as body weight per height in meter squared (kg/m²). Obesity refers to a condition whereby an otherwise healthy subject has a BMI greater than or equal to 30 kg/m², or a condition whereby a subject with at least one co-morbidity has a BMI greater than or equal to 27 kg/m². An “obese subject” is an otherwise healthy subject with a BMI greater than or equal to 30 kg/m² or a subject with at least one co-morbidity with a BMI greater than or equal 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m². The increased risks associated with obesity may occur at a lower BMI in people of Asian descent. In Asian and Asian-Pacific countries, including Japan, “obesity” refers to a condition whereby a subject with at least one obesity-induced or obesity-related co-morbidity that requires weight reduction or that would be improved by weight reduction, has a BMI greater than or equal to 25 kg/m². An “obese subject” in these countries refers to a subject with at least one obesity-induced or obesity-related co-morbidity that requires weight reduction or that would be improved by weight reduction, with a BMI greater than or equal to 25 kg/m². In these countries, a “subject at risk of obesity” is a person with a BMI of greater than 23 kg/m² to less than 25 kg/m².

In some instances, the metabolic disorder is an obesity-related metabolic disorder. The term “obesity-related disorders” encompasses disorders that are associated with, caused by, or result from obesity. Examples of obesity-related disorders include overeating and bulimia, diabetes, hypertension, elevated plasma insulin concentrations and insulin resistance, dyslipidemia, hyperlipidemia, breast, prostate, endometrial and colon cancer, heart disease, cardiovascular disorders, abnormal heart rhythms and arrhythmias, myocardial infarction, congestive heart failure, coronary heart disease, angina pectoris, cerebral infarction, cerebral thrombosis and transient ischemic attack. Other examples include pathological conditions showing reduced metabolic activity or a decrease in resting energy expenditure as a percentage of total fat-free mass. Further examples of obesity-related disorders include metabolic syndrome, also known as syndrome X, insulin resistance syndrome, type II diabetes, impaired fasting glucose, impaired glucose tolerance, inflammation, such as systemic inflammation of the vasculature, atherosclerosis, hypercholesterolemia, hyperuricaemia, as well as secondary outcomes of obesity such as left ventricular hypertrophy. Obesity-related metabolic disorders may further include, e.g., hypertension, osteoarthritis, Type II diabetes mellitus, increased blood pressure, stroke, and heart disease. Obesity-related disorders also include the liver abnormalities associated with obesity such as steatosis or non-alcoholic fatty liver disease (NAFLD) a rising cause of cirrhosis associated to obesity and metabolic syndrome. Indeed, NAFLD can present as simple steatosis or evolve towards inflammation and steatohepatitis (NASH), with a 20% risk of cirrhosis after 20 years. “Dyslipidemia” is a major risk factor for coronary heart disease (CHD). Low plasma levels of high density lipoprotein (HDL) cholesterol with either normal or elevated levels of low density (LDL) cholesterol is a significant risk factor for developing atherosclerosis and associated coronary artery disease in humans. Dyslipidemia is often associated with obesity. Additional obesity related disorders are described in, e.g., U.S. Pat. No. 8,394,969, the disclosure of which is incorporated herein by reference in its entirety.

In some instances, the metabolic disorder is diabetes. “Diabetes” refers to a group of metabolic diseases characterized by high blood sugar (glucose) levels which result from defects in insulin secretion or action, or both. Diabetes is classified according to the types of disease into insulin dependent diabetes (IDDM; type I diabetes) and non-insulin dependent diabetes (NIDDM; type II diabetes). “Type 2 diabetes” refers to one of the two major types of diabetes, the type in which the beta cells of the pancreas produce insulin, at least in the early stages of the disease, but the body is unable to use it effectively because the cells of the body are resistant to the action of insulin. In later stages of the disease the beta cells may stop producing insulin. Type 2 diabetes is also known as insulin-resistant diabetes, non-insulin dependent diabetes and adult-onset diabetes. “Type I diabetes” refers to a condition that results from an autoimmune-mediated destruction of pancreatic p cells with consequent loss of insulin production, which results in hyperglycemia. Type I diabetics require insulin replacement therapy to ensure survival. The term “diabetic disorders” may refer to complications due to diabetes. For example, complications such as retinopathy, nephropathy and neuropathy develop with angiopathy as a prime factor in diabetic individuals.

Treatment may result in various outcomes. In certain embodiments, treatment of obesity and obesity-related disorders refers to the administration of the N-lactoyl-amino acid or combinations of N-lactoyl-amino acids as described herein to reduce or maintain the body weight of an obese subject. One outcome of treatment may be reducing the body weight of an obese subject relative to that subject's body weight immediately before the administration of the compounds or combinations as described herein. Another outcome of treatment may be preventing regain of body weight previously lost as a result of diet, exercise, or pharmacotherapy and preventing weight gain from cessation of smoking. Another outcome of treatment may be decreasing the occurrence of and/or the severity of obesity-related diseases. Yet another outcome of treatment may be decreasing the risk of developing diabetes in an overweight or obese subject. The treatment may result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate. The treatment may result in weight reduction in patients in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

In some instances, the methods prevent the development of obesity or obesity-related disorders in a subject. Prevention of obesity and obesity-related disorders refers to the administration of the N-lactoyl amino acid or combinations of N-lactoyl-amino acids to reduce or maintain the body weight of a subject at risk of obesity. One outcome of prevention may be reducing the body weight of a subject at risk of obesity relative to that subject's body weight immediately before the administration of the compounds or combinations of the present invention. Another outcome of prevention may be preventing regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, Type 2 diabetes, polycystic ovary disease, cardiovascular diseases, osteoarthritis, dermatological disorders, hypertension, insulin resistance, hypercholesterolemia, hypertriglyceridemia, and cholelithiasis.

In certain embodiments, the methods reduce food intake of the subject, e.g., during and/or after treatment. By “food intake” is meant the amount of food consumed by the subject. In some instances, food intake is measured in kcal over a period of time, e.g., kcal/day. In some instances, the food intake is cumulative food intake over a period of time ranging from 1 day to 14 days, e.g., from 1 day to 10 days, from 1 day to 7 days, from 1 day to 5 days, or from 1 day to 3 days. In some instances, the food intake is average daily food intake, e.g., over a period of time ranging from 1 day to 14 days, e.g., from 1 day to 10 days, from 1 day to 7 days, from 1 day to 5 days, or from 1 day to 3 days. In some instances, the methods reduce food intake by the subject compared to (e.g., relative to) a control. In some instances, the methods reduce food intake by the subject compared to the food intake of the subject before treatment. In some instances, cumulative food intake is reduced by 10% to 90% including, e.g., by 10% to 80%, by 10% to 70%, by 10% to 60%, by 10% to 50%, by 10% to 40%, by 10% to 30%, or by 10% to 20%. In some instances, average daily food intake is reduced by 10% to 90% including, e.g., by 10% to 80%, by 10% to 70%, by 10% to 60%, by 10% to 50%, by 10% to 40%, by 10% to 30%, or by 10% to 20%.

In certain embodiments, the methods reduce the body weight of the subject, e.g., during and/or after treatment. In some instances, the methods reduce the body weight of the subject compared to (e.g., relative to) a control. In some instances, the methods reduce the body weight of the subject compared to the body weight of the subject before treatment. In some instances, the methods reduce the average body weight of the subject over a period of time ranging, e.g., from 1 day to 14 days, e.g., from 1 day to 10 days, from 1 day to 7 days, from 1 day to 5 days, or from 1 day to 3 days. In some instances, the methods reduce the average body weight of the subject compared to a control. In some instances, the methods reduce the average body weight of the subject compared to the average body weight of the subject before treatment. In some instances, body weight is reduced by 1% to 50% including, e.g., by 1% to 40%, by 1% to 30%, by 1% to 20%, or by 1% to 10%. In some instances, average body weight is reduced by 1% to 50% including, e.g., by 1% to 40%, by 1% to 30%, by 1% to 20%, or by 1% to 10%.

In certain embodiments, the methods improve glucose regulation in a subject, e.g., during and/or after treatment. For example, the methods may improve the body's ability to regulate glucose. The term “glucose regulation” or “regulation of glucose metabolism” as used herein refer to processes by which a cell, tissue, organ, organ system, or whole organism maintains glucose homeostasis by altering, e.g., increasing or decreasing, specific processes of glucose metabolism. Glucose metabolism or glucose metabolic processes encompass processes involving glucose synthesis, processing, transport, uptake, utilization, or storage, and includes gluconeogenesis and glycolysis. Specific aspects of glucose metabolism and regulation include expression of glucose transporters or enzymes which facilitate movement of glucose across a cell membrane and retention or secretion of glucose by a cell; alteration in expression and/or activity of enzymes involved in glucose utilization or formation, including, e.g., glycolytic and gluconeogenic enzymes; and alteration of glucose distribution within body or culture fluids, including, e.g., interstitial (i.e. extracellular) and intracellular fluids, blood, urine, and the like.

In some embodiments, the methods improve glucose homeostasis in the subject, e.g., during and/or after treatment. The term “glucose homeostasis” refers to maintenance of normal glucose levels, e.g., normal blood glucose levels, in an organism. In some instances, the methods improve glucose homeostasis in the subject compared to (e.g., relative to) a control. In some instances, the methods improve glucose homeostasis in the subject compared to the glucose homeostasis in the subject before treatment. In some instances, the methods improve glucose clearance, e.g., from circulation, in the subject compared to a control. In some instances, the methods improve glucose clearance in the subject compared to the glucose clearance in the subject before treatment. Improved glucose clearance may include increased glucose clearance. Increased glucose clearance may reduce blood glucose levels. In some instances, glucose clearance is improved by 1% to 50% including, e.g., by 1% to 40%, by 1% to 30%, by 1% to 20%, or by 1% to 10%.

In some embodiments, the methods reduce adipose tissue mass (e.g., the amount of adipose tissue or fat) in the subject. The term “adipose tissue” refers to fat including, e.g., the connective tissue that stores fat. Adipose tissue contains multiple regenerative cell types, including, e.g., adipose derived stem cells (ASCs) and endothelial progenitor and precursor cells. Types of adipose tissue of interest include, but are not limited to, white adipose tissue and brown adipose tissue. In some instances, the methods reduce adipose tissue mass compared to a control. In some instances, the methods reduce adipose tissue mass in a subject compared to the adipose tissue mass in the subject before treatment. In some instances, the methods reduce adipose tissue by 1% to 50% including, e.g., by 1% to 40%, by 1% to 30%, by 1% to 20%, or by 1% to 10%. In some instances, the methods reduce adipose tissue by 10% to 50% including, e.g., by 10% to 40%, by 10% to 30%, by 10% to 20%, by 20% to 50%, by 30% to 50%, or by 40% to 50%. In some instances, the methods reduce the amount of white fat compared to a control. In some instances, the methods reduce the amount of white fat in a subject compared to the amount of white fat in the subject before treatment. In some instances, the methods reduce white fat by 1% to 50% including, e.g., by 1% to 40%, by 1% to 30%, by 1% to 20%, or by 1% to 10%. In some instances, the methods reduce white fat by 10% to 50% including, e.g., by 10% to 40%, by 10% to 30%, by 10% to 20%, by 20% to 50%, by 30% to 50%, or by 40% to 50%. In some instances, the methods reduce the amount of brown fat compared to a control. In some instances, the methods reduce the amount of brown fat in a subject compared to the amount of brown fat in the subject before treatment. In some instances, the methods reduce brown fat by 1% to 50% including, e.g., by 1% to 40%, by 1% to 30%, by 1% to 20%, or by 1% to 10%. In some instances, the methods reduce brown fat by 10% to 50% including, e.g., by 10% to 40%, by 10% to 30%, by 10% to 20%, by 20% to 50%, by 30% to 50%, or by 40% to 50%. In some instances, the methods reduce the amount of smaller epididymal fat compared to a control. In some instances, the methods reduce the amount of smaller epididymal fat in a subject compared to the amount of smaller epididymal fat in the subject before treatment. In some instances, the methods reduce smaller epididymal fat by 1% to 50% including, e.g., by 1% to 40%, by 1% to 30%, by 1% to 20%, or by 1% to 10%. In some instances, the methods reduce smaller epididymal fat by 10% to 50% including, e.g., by 10% to 40%, by 10% to 30%, or by 10% to 20%. In some instances, the methods reduce the amount of subcutaneous inguinal fat compared to a control. In some instances, the methods reduce the amount of subcutaneous inguinal fat in a subject compared to the amount of subcutaneous inguinal fat in the subject before treatment. In some instances, the methods reduce subcutaneous inguinal fat by 1% to 50% including, e.g., by 1% to 40%, by 1% to 30%, by 1% to 20%, or by 1% to 10%. In some instances, the methods reduce subcutaneous inguinal fat by 10% to 50% including, e.g., by 10% to 40%, by 10% to 30%, by 10% to 20%, by 20% to 50%, by 30% to 50%, or by 40% to 50%.

The “control,” as used herein in its conventional sense, may be any suitable control. In some embodiments, the control includes a subject, e.g., a subject with a metabolic disorder, to whom an effective amount of an N-lactoyl-amino acid has not been administered. The control may be a subject that has the same metabolic disorder(s) and/or associated conditions as the treated subject. In some embodiments, the control includes a subject to whom an effective amount of an N-lacotyl-amino acid is not administered, where the subject has a metabolic disorder or a combination of metabolic disorders and/or associated conditions that match those of the subject to whom the effective amount is administered. In some instances, the control subject has characteristics (e.g., age, sex, height, weight, race, diet, etc.) that are shared by the treated subject. In some instances, the outcomes for a subject as described herein (related to, e.g., food intake by the subject, body weight of the subject, glucose homeostasis in the subject, adipose tissue mass of the subject, etc.) are measured relative to the subject before treatment.

Embodiments of the methods can be practiced on any suitable subject. A subject of the present invention may be a “mammal” or “mammalian”, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some instances, the subjects are humans. The methods may be applied to human subjects of both genders and at any stage of development (i.e., neonates, infant, juvenile, adolescent, adult), where in certain embodiments the human subject is a juvenile, adolescent or adult.

### Pharmaceutical Formulations

As summarized above, pharmaceutical formulations or pharmaceutical compositions are provided. A pharmaceutical formulation may include an N-lactoyl-amino acid (or a combination of N-lactoyl-amino acids) or a pharmaceutically acceptable salt thereof, and one or more of pharmaceutically acceptable carriers or excipients. In some instances, a pharmaceutical formulation includes an amount of an N-lactoyl-amino acid effective to treat a metabolic disease; and an excipient. The N-lactoyl-amino acid may be any suitable N-lactoyl-amino acid or combination of N-lactoyl amino acids as described herein. In some instances, the N-lactoyl-amino acid is N-lactoyl-phenylalanine. The amount of N-lactoyl-amino acid, e.g., effective amount, may be any suitable amount according to any of the embodiments described herein. The pharmaceutical formulations may be administered in combination with any of the therapies (e.g., therapies for treating a metabolic disorder) as described herein.

The pharmaceutical formulation may be formulated for administration by any suitable means. In certain embodiments, the composition is formulated for administration orally, intradermally, intramuscularly, parenterally, intravenously, intra-arterially, intracranially, subcutaneously, intraorbitally, intraventricularly, intraspinally, intraperitoneally, or intranasally. The pharmaceutical formulations or compositions can be formulated into various dosage forms, including tablets, powders, fine granules, granules, dry syrups, capsules, liquid compositions, etc. In some instances, the pharmaceutical formulation is a capsule or tablet. In some instances, the pharmaceutic formulation is a parenteral formulation. In some instances, the pharmaceutical formulation is an intraperitoneal formulation.

Additives and diluents normally utilized in the pharmaceutical arts can optionally be added to the pharmaceutical formulation. These include thickening, granulating, dispersing, flavoring, sweetening, coloring, and stabilizing agents, including pH stabilizers, other excipients, anti-oxidants (e.g., tocopherol, BHA, BHT, TBHQ, tocopherol acetate, ascorbyl palmitate, ascorbic acid propyl gallate, and the like), preservatives (e.g., parabens), and the like. Exemplary preservatives include, but are not limited to, benzylalcohol, ethylalcohol, benzalkonium chloride, phenol, chlorobutanol, and the like. Some useful antioxidants provide oxygen or peroxide inhibiting agents for the formulation and include, but are not limited to, butylated hydroxytoluene, butylhydroxyanisole, propyl gallate, ascorbic acid palmitate, α-tocopherol, and the like. Thickening agents, such as lecithin, hydroxypropylcellulose, aluminum stearate, and the like, may improve the texture of the formulation.

A container for holding the N-lactoyl-amino acid formulation or N-lactoyl-amino acid pharmaceutical composition may be configured to hold any suitable volume of the N-lactoyl-amino acid formulation or composition. In some cases, the size of the container may depend on the volume of N-lactoyl-amino acid composition to be held in the container. In certain embodiments, the container may be configured to hold an amount of N-lactoyl-amino acid composition ranging from 0.1 mg to 1000 mg, such as from 0.1 mg to 900 mg, such as from 0.1 mg to 800 mg, such as from 0.1 mg to 700 mg, such as from 0.1 mg to 600 mg, such as from 0.1 mg to 500 mg, such as from 0.1 mg to 400 mg, or 0.1 mg to 300 mg, or 0.1 mg to 200 mg, or 0.1 mg to 100 mg, 0.1 mg to 90 mg, or 0.1 mg to 80 mg, or 0.1 mg to 70 mg, or 0.1 mg to 60 mg, or 0.1 mg to 50 mg, or 0.1 mg to 40 mg, or 0.1 mg to 30 mg, or 0.1 mg to 25 mg, or 0.1 mg to 20 mg, or 0.1 mg to 15 mg, or 0.1 mg to 10 mg, or 0.1 mg to 5 mg, or 0.1 mg to 1 mg, or 0.1 mg to 0.5 mg. In certain embodiments, the container is configured to hold an amount of N-lactoyl-amino acid composition ranging from 0.1 g to 10 g, or 0.1 g to 5 g, or 0.1 g to 1 g, or 0.1 g to 0.5 g. In certain instances, the container is configured to hold a volume (e.g., a volume of a liquid N-lactoyl-amino acid composition) ranging from 0.1 ml to 200 ml. For instance, the container may be configured to hold a volume (e.g., a volume of a liquid) ranging from 0.1 ml to 1000 ml, such as from 0.1 ml to 900 ml, or 0.1 ml to 800 ml, or 0.1 ml to 700 ml, or 0.1 ml to 600 ml, or 0.1 ml to 500 ml, or 0.1 ml to 400 ml, or 0.1 ml to 300 ml, or 0.1 ml to 200 ml, or 0.1 ml to 100 ml, or 0.1 ml to 50 ml, or 0.1 ml to 25 ml, or 0.1 ml to 10 ml, or 0.1 ml to 5 ml, or 0.1 ml to 1 ml, or 0.1 ml to 0.5 ml. In certain instances, the container is configured to hold a volume (e.g., a volume of a liquid N-lactoyl-amino acid composition) ranging from 0.1 ml to 200 ml.

The shape of the container may also vary. In certain cases, the container may be configured in a shape that is compatible with the assay and/or the method or other devices used to perform the assay. For instance, the container may be configured in a shape of typical laboratory equipment used to perform the assay or in a shape that is compatible with other devices used to perform the assay. In some instances, the container is a liquid container. In some embodiments, the liquid container is a vial or a test tube. In certain cases, the liquid container is a vial. In certain cases, the liquid container is a test tube. In some instances, the container is a blister pack.

As described above, embodiments of the container can be compatible with the N-lactoyl-amino acid composition. Examples of suitable materials for the containers include, but are not limited to, glass and plastic. For example, the container may be composed of glass, such as, but not limited to, silicate glass, borosilicate glass, sodium borosilicate glass (e.g., PYREX™), fused quartz glass, fused silica glass, and the like. Other examples of suitable materials for the containers include plastics, such as, but not limited to, polypropylene, polymethylpentene, polytetrafluoroethylene (PTFE), perfluoroethers (PFE), fluorinated ethylene propylene (FEP), perfluoroalkoxy alkanes (PFA), polyethylene terephthalate (PET), polyethylene (PE), polyetheretherketone (PEEK), and the like.

In some embodiments, the container may be sealed. That is, the container may include a seal that substantially prevents the contents of the container from exiting the container. The seal of the container may also substantially prevent other substances from entering the container. For example, the seal may be a water-tight seal that substantially prevents liquids from entering or exiting the container, or may be an air-tight seal that substantially prevents gases from entering or exiting the container. In some instances, the seal is a removable or breakable seal, such that the contents of the container may be exposed to the surrounding environment when so desired, e.g., if it is desired to remove a portion of the contents of the container. In some instances, the seal is made of a resilient material to provide a barrier (e.g., a water-tight and/or air-tight seal) for retaining a sample in the container. Particular types of seals include, but are not limited to, films, such as polymer films, caps, etc., depending on the type of container. Suitable materials for the seal include, for example, rubber or polymer seals, such as, but not limited to, silicone rubber, natural rubber, styrene butadiene rubber, ethylene-propylene copolymers, polychloroprene, polyacrylate, polybutadiene, polyurethane, styrene butadiene, and the like, and combinations thereof. For example, in certain embodiments, the seal is a septum pierceable by a needle, syringe, or cannula. The seal may also provide convenient access to a sample in the container, as well as a protective barrier that overlies the opening of the container. In some instances, the seal is a removable seal, such as a threaded or snap-on cap or other suitable sealing element that can be applied to the opening of the container. For instance, a threaded cap can be screwed over the opening before or after a sample has been added to the container.

### Utility

The subject methods and formulations find use in applications, e.g., clinical applications, involving metabolic disorders and one or more conditions associated with metabolic disorders. In some embodiments, the methods and formulations find use in applications where it is desirable to treat a metabolic disorder and one or more conditions associated with metabolic disorders including, e.g., obesity, an obesity related disorder, diabetes, etc. In some instances, the methods and formulations find use in applications where it is desirable to prevent the development or occurrence of a metabolic disorder and one or more conditions associated with metabolic disorders. In certain embodiments, the methods and formulations find use in applications where weight loss for a subject is desirable. In certain embodiments, the methods and formulations find use in applications where improving glucose homeostasis in a subject is desirable. In certain embodiments, the methods and formulations find use in applications where it is desirable to induce a physical activity associated outcome in a subject. In certain embodiments, the methods and formulations find use in applications where it is desirable to treat a nervous system disorder. The methods and formulations may also find use in combination with other therapies and treatments for any of the disorders and associated conditions described herein.

The following example(s) is/are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### Example 1: A Lactate-Derived Exercise-Inducible Metabolite that Suppresses Food Intake and Obesity

### Materials and Methods

Cell line cultures. All cell lines were obtained from ATCC and grown at 37° C. with 5% CO₂. RAW 264.7, HEK293T, HEK293A, C2C12, F442A, 3T3-L1, Caco2, and SW48 were grown in Dulbecco's modified Eagle's medium (DMEM) with 10% fetal bovine serum (FBS) and penicillin/streptomycin (pen/strep). AML-12 cells were grown in DMEM with 10% FBS and pen/strep supplemented with Insulin-Transferrin-Selenium. T84 cells were grown in Dulbecco's modified Eagle's medium/nutrient mixture F-12 (DMEM/F-12) with 5% FBS and pen/strep.

General animal information. Animal experiments were performed according to procedure approved by the Stanford University Administrative Panel on Laboratory Animal Care (APLAC). Mice were maintained in 12-hr light-dark cycles at 22° C. and ˜50% relative humidity and fed a standard irradiated rodent chow diet. Where indicated, high-fat diet (D12492, Research Diets 60% kcal from fat) was used. C57BL/6J (stock no. 000664) and C57BL/6J DIO mice (stock no. 380050) were purchased from Jackson Laboratory. C57BL/6NCrl (stock no. 027) mice were purchased from Charles River Laboratory. Whole body CNDP2 knockout mice (catalog number, C57BL/6NCrl-Cndp2em1(IMPC)Mbp/Mmucd, RRID: MMRRC_043492-UCD) were obtained from the Mutant Mouse Regional Resource Center, a NCRR-NIH funded strain repository. For in vivo injection of mice with compounds including Lac-Phe, lactate, and phenylalanine, compounds were dissolved in 18:1:1 (by volume) of saline/Kolliphor EL (Sigma Aldrich)/DMSO. Compounds were administered to mice daily via intraperitoneal injections at 5 μl/g body weight at the indicated doses. For all injection experiments, mice were mock injected with the vehicle for 3-5 days until body weights have stabilized. For glucose tolerance tests, mice were fasted for 6 hours then injected with glucose at 10 μl/g body weight. A dose of 1 g/kg was used for the GTT of vehicle and Lac-Phe treated obese mice.

Chemicals. L-Phenylalnine (AAA-1323814) was purchased from Fisher Scientific, sodium L-lactate (L7022) was purchased from Sigma. The synthesis of non-commercially available Lac-Phe is described below.

Synthesis of Lac-Phe (N-lactoyl phenylalanine). Sodium L-lactate (1.2 eq.) was dissolved in dichloromethane (0.2 M) and treated with 3-[bis(dimethylamino)methyliumyl]-3H-benzotriazol-1-oxide hexafluorophosphate (HBTU, 1.2 eq.) at 0° C. under argon. After 15 minutes, phenylalanine methyl ester hydrochloride (1.0 eq.) and N, N-diisopropylethylamine (3.0 eq.) in dichloromethane (0.2 M) was added to the mixture. The reaction was stirred for 16 hours under argon at ambient temperature. One third of the solvent was removed and the dichloromethane solution washed with 5% HCl, 5% NaHCO₃, and saturated NaCl solutions. The organic layer was dried (MgSO₄), filtered, and concentrated. The resulting crude product was purified by column chromatography, eluting with ethyl acetate/hexane to afford the N-Phenylalanine methyl ester. The above ester (1.0 eq.) was dissolved in THF (0.5 M) and treated with lithium hydroxide monohydrate (2.0 eq.) in water (0.5 M). The solution was stirred at ambient temperature for 2 hours, and the solvent removed. The resulting residue was dissolved in dichloromethane and acidified by 5% HCl to pH 3. The resulting mixture was extracted with ethyl acetate three times and the combined organic layers washed with saturated NaCl solution. The organic layer was dried (MgSO₄), filtered, and concentrated. The resulting crude product was purified with recrystallization with ethyl acetate/hexane to give the N-lactoyl Phenylalanine as a white powder. ¹H-NMR (400 MHz, D₂O) δ 7.3-7.2 (m, 5H), 4.13 [dd, 1H], 4.10 [q, 1H], 3.21 [dd, 1H], 2.98 [dd, 1H], 1.10 [d, 3H]. LC/MS (m/z): 236.093 [M-H]⁻

Mouse running protocols. For mouse running studies a 6 lane Columbus Instruments animal treadmill (product 1055-SRM-D65) was used. Prior to treadmill running, mice were acclimated to the treadmill for 5 minutes. For acute, exercise studies to exhaustion, treadmill running began at a speed of 7.5 m/min and a 4° incline. Every three minutes the speed and incline were increased by 2.5 m/min and 2°, respectively. Once a speed of 40 m/min and incline of 30° was reached, both parameters were kept constant until mice reached exhaustion. Exhaustion was defined as when the mice remained on the shocker at the back of the treadmill for longer than 5 seconds. For chronic running experiments with WT and CNDP2-KO mice, mice were exercised 5 days/week, Monday through Friday while on high fat diet (60% kcal from fat) and were allowed to rest the remaining 2 days/week. Treadmill running was performed at a constant 5° incline and began at a speed of 6 m/min. Speed was increased by 2 m/min every 5 minutes until a maximum speed of 30 m/min. Mice were stopped upon reaching exhaustion as described above and run times were normalized between the two groups of mice.

Preparation of plasma samples for LC-MS analysis. Plasma was collected from mice via a submandibular bleed into lithium heparin tubes (BD, 365985) and immediately transferred onto ice. The blood was centrifuged at 4° C. at 5000 rpm for 5 min and the top layer of plasma was aliquoted and frozen at −80° C. To extract polar metabolites from plasma for LC-MS analysis, 150 ul of a 2:1 mixture of acetonitrile/methanol was added to 50 μl of plasma. The mixture was centrifuged at 4° C. for 10 min at 15,000 rpm and the supernatant was transferred to a LC-MS vial.

Untargeted measurements of metabolites by LC-MS. Untargeted metabolomics measurements were performed on an Agilent 6520 Quadrupole Time-of-Flight (Q-TOF) LC/MS. Mass spectrometry analysis was performed using electrospray ionization (ESI) in negative mode. The dual ESI source parameters were set as follows, the gas temperature was set at 250° C. with a drying gas flow of 12 l/min and the nebulizer pressure at 20 psi. The capillary voltage was set to 3500 V and the fragmentor voltage set to 100 V. Separation of polar metabolites was conducted on a Luna 5 μm NH2 100 Å LC column (Phenomenex 00B-4378-E0) with normal phase chromatography. Mobile phases were as follows: Buffer A, 95:5 water/acetonitrile with 0.2% ammonium hydroxide and 10 mM ammonium acetate. Buffer B, acetonitrile. The LC gradient started at 100% B with a flow rate of 0.2 ml/min from 0-2 min. The gradient was then increased linearly to 50% A/50% B at a flow rate of 0.7 ml/min from 2-20 minutes. From 20-25 minutes the gradient was maintained at 50% A/50% B at a flow rate of 0.7 ml/min.

Targeted measurements of Lac-AAs. Targeted measurements of performed on an Agilent 6470 Triple Quadrupole (QQQ) LC/MS. Mass spectrometry analysis was performed using electrospray ionization (ESI) in negative mode. The AJS ESI source parameters were set as follows, the gas temperature was set at 250° C. with a gas flow of 12 l/min and the nebulizer pressure at 25 psi. The sheath gas temperature was set to 300° C. with the sheath gas flow set at 12 l/min. The capillary voltage was set to 3500 V. Separation of polar metabolites was performed as described above in the untargeted metabolomics section. Multiple reaction monitoring (MRM) was performed for the indicated metabolites with the listed dwell times, fragmentor voltage, collision energies, cell accelerator voltages, and polarities.

| | | | | | | | | Cell | |
|---|---|---|---|---|---|---|---|---|---|
| | Precursor | MS1 | Product | MS2 | | | Collision | Accelerator | |
| Compound | Ion | Res | Ion | Res | Dwell | Fragmentor | Energy | Voltage | Polarity |
| N-Lactoyl | 236.1 | Unit | 88 | Unit | 50 | 135 | 10 | 5 | Negative |
| Phe | | | | | | | | | |
| Arachidonate | 303.2 | Unit | 303.2 | Unit | 50 | 135 | 0 | 5 | Negative |
| Oleate | 281.2 | Unit | 281.2 | Unit | 50 | 135 | 0 | 5 | Negative |
| Lactate | 89.0 | Unit | 89.0 | Unit | 50 | 135 | 0 | 5 | Negative |

Differentiation of cell lines. C2C12 cells were plated in 12-wells and grown until 80-90% confluence. Cells were incubated in DMEM with 2% horse serum and pen/strep. Culture media was changed every 2 days for 4-5 days. F442A cells were plated in 12-wells and grown until 60-70% confluence. Cells were differentiated by addition of 5 μg/ml insulin and 1 μM rosiglitazone into complete media. Culture media was changed every 2 days for 7-8 days. 3T3-L1 cells were plated in 12-wells and grown until 80-90% confluence. Differentiation was initiated by addition of a cocktail containing 5 μg/ml insulin, 5 μM dexamethasone, 250 μM isobutylmethylxanthine, and 1 μM rosiglitazone for 2 days. After induction, cells were maintained in 5 μg/ml insulin and 1 μM rosiglitazone for the remaining 4-6 days.

Generation of CNDP2-KO RAW 264.7 cells. The plentiCRISPRv2 system developed by the Zhang lab was used to generate the CNDP2-KO RAW 264.7 cell line. The sgRNA used was 5′-CAGTGAAATGAGATCCGTCA-3′(SEQ ID NO:01). Following Zhang lab protocols, oligonucleotides for the sgRNA and reverse complement sequences were synthesized and cloned into the plentiCRISPRv2 vector (Forward oligo, 5′-CACCGCAGTGAAATGAGATCCGTCA-3′ (SEQ ID NO:02); reverse oligo, 5′-AAACTGACGGATCTCATTTCACTG C-3′ (SEQ ID NO:03)). Lentivirus particles were generated in the HEK293T cell line using polyfect for the co-transfection of the cloned plentiCRISPRv2 plasmid with the viral packing psPAX2 plasmid, and viral envelope pMD2.G plasmid. A parental plentiCRISPRv2 plasmid was used as a control. Lentiviral supernatants were harvested after 24 hours and filtered through a 0.45 uM filter. The supernatant was then mixed in a 1 to 1 ratio with polybrene to a final concentration of 8 ug/ml polybrene. This mixture was added to RAW 264.7 cells at 40-50% confluence in 6-well plates. Transduced cells were transferred to a 10 cm plate followed by selection with 5 ug/ml of puromycin for 3-6 days.

Primary peritoneal macrophage isolation. Mice were injected with 2 ml of 3% brewer thioglycollate medium (Fisher, B111716) and the macrophage elicitation allowed to proceed for 3 days. For the macrophage isolation, mice were euthanized with CO₂ and the abdominal skin peeled back to expose the peritoneal wall. 10 ml of ice-cold calcium and magnesium free DPBS was injected into the peritoneal cavity, taking care not to puncture the organs. The mice were gently massaged and the fluid aspirated from the peritoneum using the same syringe and needle. The fluid was dispensed into a 50 ml falcon tube on ice. Cells were then centrifuged at 4° C. for 10 minutes at 400×g. The supernatant was discarded and the cells re-suspended in cold DMEM/F-12 containing 10% FBS and pen/strep. Cells were allowed to adhere in the incubator for at least 2 hours then washed three times in warm PBS to remove non-adherent cells. Fresh media was added to the cells and experiments performed within 1-2 days after the isolation.

In vitro Lac-Phe Production assay. Cells were plated in 12-well plates at 70-80% confluence. The next day, cells were washed two times with PBS and incubated in 0.5 ml serum free media. After overnight incubation, 400 μl of media was removed and 20 μl of 1 M hydrochloride added to acidify the media and protonate Lac-Phe. 400 μl of ethyl acetate was added into each sample and vortexed for 30 seconds to extract Lac-Phe into the organic layer. 300 μl from the top layer was transferred to a new Eppendorf tube and dried down under a stream of nitrogen. The residue was re-suspended in 100 ul of an 80:20 mixture of acetonitrile/water. Cells were kept on ice to harvest the lysate. 150 μl of PBS was added into each well and the cells scraped into an Eppendorf tube. This step was repeated again to ensure all cells have been harvested. Cells were then centrifuged at 4° C. for 10 minutes at 2,000×g and the supernatant removed to obtain the cell pellet. 100 μl of a 2:1:1 mixture of acetonitrile/methanol/water mixture was used to lyse the cells and precipitate large proteins. The mixture was centrifuged at 4° C. for 10 minutes at 15,000 rpm and the supernatant was transferred to a LC-MS vial.

Western blot analysis. Cells were collected and lysed by sonication in RIPA buffer containing 1:100 HALT protease inhibitor. Cell lysates were centrifuged at 4° C. for 10 minutes at 13,000 rpm to remove residual cell debris. Protein concentrations of the supernatant were normalized using the Pierce BCA protein assay kit and combined with 4×NuPAGE LDS Sample Buffer with 10 mM DTT. Samples were then boiled for 10 minutes at 95° C. Prepared samples were run on a NuPAGE 4-12% Bis-Tris gel then transferred to nitrocellulose membranes. Blots were blocked for 30 minutes at room temperature in Odyssey blocking buffer. Primary antibodies (rabbit anti-CNDP2 and rabbit anti-Beta-Tubulin) were added to Odyssey blocking buffer at a ratio of 1:1000. Blots were incubated in the indicated primary antibodies overnight while shaking at 4° C. The following day, blots were washed 3 times with PBS-T, 10 minutes each before staining with the secondary antibody for 1 hour at room temperature. The secondary antibody used was a goat anti-rabbit antibody diluted in blocking buffer to a ratio of 1:10,000. Following secondary antibody staining, the blot was washed 3 times with PBS-T before being imaged with the Odyssey CLx Imaging System.

Generation of CNDP2 KO animals. CNDP2 KO and wild type animals were generated via heterozygous breeding crosses. Genotyping was performed as follows: Tail clippings were obtained from littermates and boiled for 30 minutes at 95° C. in 100 μl of 50 mM NaOH to extract genomic DNA. The solution was neutralized by adding 21 μl of 0.5 M Tris (pH 7.2). PCR reactions were performed by using primers for either the CNDP2 WT allele (Forward: 5′-CAGATGGCTCGGAGATACCAC-3′ (SEQ ID NO:04), Reverse: 5′-TTCCCGCTCCACCAAGGTGAAG-3′ (SEQ ID NO:05)) or CNDP2 KO allele (Forward: 5′-GCTCTGTAAGGGAAAGAGATGACCC-3′ (SEQ ID NO:06), Reverse: 5′-AATAGGACATACCCAGTTCTGTGAGG-3′ (SEQ ID NO:07)). The Promega GoTaq master mix was used for the PCR reaction. Each 25 μl reaction consisted of 12.5 μl of the promega master mix (M7122), 2.5 μl of a 10 μM mixture of forward and reverse primers, 2 μl of genomic DNA, and 8 μl of ultrapure water. The thermocycling program on BioRad C1000 Touch Thermo Cycler began with an initial 30 seconds at 95° C., followed by cycles of 30 seconds at 98° C., 30 seconds at 58° C., and 45 seconds at 72° C., followed by 5 minutes at 72° C. and finally held at 4° C. PCR reactions for WT primers consisted of 30 cycles while PCR reactions for KO primers consisted of 48 cycles. Samples were run on a 2% agarose gel with 0.2 mg/ml EtBr. WT alleles are expected to yield a PCR product 160 base pairs in size while KO alleles are expected to yield PCR products that are 440 base pairs in size.

Slice electrophysiology studies. Several genetic mouse strains were used for electrophysiological recordings from identified neural populations. These included AgRP-IRES-Cre/Rosa26-LSL-tdTOMATO mice for recordings from AgRP neurons in the arcuate nucleus of hypothalamus (ARH); POMC-CreER/Rosa26-LSL-tdTOMATO mice (with 200 mg/kg tamoxifen induction at 8-12 weeks) were used for recordings from POMC neurons in the ARH; SF1-Cre/Rosa26-LSL-tdTOMATO mice were used for recordings from SF1 neurons in the ventromedial hypothalamus (VMH); TPH2-CreER/Rosa26-LSL-tdTOMATO mice (with 200 mg/kg tamoxifen induction at 8-12 weeks) were used for recording from 5-HT neurons in the dorsal raphe nucleus (DRN). Mice (males and females, 8-16 weeks old) were anesthetized with isoflurane and transcardially perfused with a modified ice-cold sucrose-based cutting solution (pH 7.4; containing 10 mM NaCl, 25 mM NaHCO₃, 195 mM sucrose, 5 mM glucose, 2.5 mM KCl, 1.25 mM NaH2PO₄, 2 mM sodium pyruvate, 0.5 mM CaCl₂, and 7 mM MgCl₂, bubbled continuously with 95% O₂ and 5% CO₂). The mice were then decapitated, and the entire brain was removed and immediately submerged in the cutting solution. Coronal slices (220 μm) were cut with a Microm HM 650V vibratome (Thermo Scientific). Brain slices containing the desired region (ARH, VMH, or DRN) were collected for each corresponding mouse, and recordings were made at levels throughout this brain region. The slices were recovered for ˜30 minutes at 32° C. and then maintained at room temperature for another 1 hour in oxygenated (95% O₂ and 5% CO₂) artificial cerebrospinal fluid (ACSF, pH 7.4; containing 126 mM NaCl, 2.5 mM KCl, 2.4 mM CaCl₂, 1.2 mM NaH₂PO₄, 1.2 mM MgCl₂, 11.1 mM glucose, and 21.4 mM NaHCO₃) before recording.

Slices were transferred to the recording chamber at 32° C. and perfused continuously with oxygenated ACSF at a flow rate of 1.8-2.0 ml/min. Slices were allowed to equilibrate for at least 5 min before recording. tdTOMATO-labeled neurons in the ARH, VMH or DRN were visualized using epifluorescence and infrared-differential interference contrast (IR-DIC) imaging on an upright microscope (Eclipse FN-1, Nikon) equipped with a moveable stage (MP-285, Sutter Instrument). Patch pipettes with resistances of 3-5 M Ow were filled with intracellular solution (pH 7.3) containing 128 mM potassium gluconate, 10 mM KCl, 10 mM HEPES, 0.1 mM EGTA, 2 mM MgCl₂, 0.05 mM GTP (sodium salt), and 0.05 mM ATP (magnesium salt). Recordings were made using a MultiClamp 700B amplifier (Axon Instruments), sampled using Digidata 1440A, and analyzed offline with pClamp 10.3 software (Axon Instruments). Series resistance was monitored during the recording, and the values were generally <10 MO and were not compensated. The liquid junction potential was monitored and corrected. Data were excluded if the series resistance exceeding 20% change during the experiment or without overshoot for action potential. Currents were amplified, filtered at 1 kHz, and digitized at 10 kHz. Current clamp was engaged to test neural firing frequency and resting membrane potential (RM) at the baseline and after puff delivery of Lac-Phe (5 s at the various concentrations indicated in the figures). To ensure each recorded neuron receive same amount of Lac-Phe, the neurons located on the surface of the slice were selected to record and the puff pipette was always put at a 100 μm horizontal and 100 μm vertical distance from the recorded neurons. The puff strength was maintained at a same level using a repeatable pressure pulse system (Picospritzer Ill, Parker). In some experiments, the ACSF solution containing 1 μM tetrodotoxin (TTX, Tocris), 50 μM bicuculline (Tocris), 20 μM DNQX (Tocris) and 50 μM D-AP5 (Tocris) was used to block the majority of presynaptic inputs. Each neuron was recorded for at least 1 minute at baseline and only the neurons with stable baseline were used to test the Lac-Phe treatment. The values of RM and firing frequency were averaged in baseline and in a 1 minute range containing the point with the maximal change in resting membrane potential after Lac-Phe puff in POMC, SF1 and 5-HT neurons. For AgRP neurons, the values of RM and firing frequency were averaged in 40 seconds at each time point indicated in the figures. A neuron was considered depolarized or hyperpolarized if a change in membrane potential was at least 2 mV, whereas values between a 2 mV was defined as “irresponsive”.

Human exercise study—single bout of acute treadmill running. This study was conducted by the Snyder Lab at Stanford University with a detailed description of the experiment model and subject details in reference #. Briefly, 36 healthy research participants were enrolled and consented to participate in the exercise study approved by the Stanford University Institutional Review Board (IRB 23602). Participants arrived at the test facility having fasted overnight (10-12 hours) and underwent symptom-limited cardiopulmonary exercise (CPX) testing on ramp-treadmills. Protocols were individualized to the fitness level of each participant and participants were encouraged to exercise until full maximal exercise capacity which lasted between 8-12 minutes. Blood was collected intravenously from the upper forearm before exercise as well as 2 min, 15 min, 30 min, and 1 hr post-exercise. The blood plasma was isolated and analyzed on an untargeted metabolomics platform.

Research design and methods for human endurance, sprint, and resistance training. Eight young healthy men were recruited to the study. The participants were 26.5±3.7 years, normal weight (BMI 23.5±2.1 kg/m2), non-smokers and not participating in regular physical activity (s 1 session/week) with a fitness level at 42.6±4.2 ml/min/kg. Prior to participation in any experimental practice, subjects were given written and oral information regarding potential risk of participation. Informed content was obtained from all subjects, in accordance with the Declaration of Helsinki II. The study was approved by the regional ethics committee in Denmark (Journal number: H-18051389). The subjects arrived at the test facilities in the morning (08:00 a.m.), after an overnight fast (10 hours fast). Body composition was measured by dual-energy x-ray absorptiometry (Lunar DPX-IQ DEXA Scanner, Lunar Corporation, WI, USA). Maximal oxygen uptake (VO2 peak) was measured with an incremental ramp test on a Monark Ergomedic 893E bicycle (Monark, Sweden), to evaluate training status. The test was comprised of 5 minutes at 100 W and 5 minutes at 150 W, followed by a 25 W increase per min until exhaustion. Expired air was collected during the test, using an online gas analyzer (CareFusion, MasterScreen-CPX, Germany). Prior to each trial, subjects were asked to refrain from strenuous physical activity for a minimum of 48 hours. The day before each trial, a standardized diet (60 E % carbohydrate, 25 E % fat, 15 E % protein) was given in weighted portions. Calculation of energy requirement was based on the WHO formula (“Human Energy Requirements: Report of a Joint FAO/WHO/UNU Expert Consultation.,” 2005) and daily physical activity level (PAL). On the morning of the experimental days, the subjects arrived in the fasted state at 08:00 a.m. by car or public transportation to avoid redundant physical activity. A venflon catheter (BD Venflon™ Pro Safety, Helsingborg, Sweden) was inserted in an antecubital vein for blood sampling at rest, acutely after exercise (0), and during recovery from exercise (15, 60, 120 and 180 minutes) After centrifugation, plasma was pipetted into aliquots (200 μl) and stored at −80° C. until further analysis. All subjects underwent three identical experimental trials, only separated by the exercise modalities performed which were: 1) an endurance exercise trial (END), 2) a sprint exercise trial (SPT) and 3) a resistance exercise trial (RES). The trials were performed in a randomized order, and each trial was separated by at least 10 days. Endurance exercise trial (END). The END trial consisted of 90 minutes of continuous cycling at 55% VO2 peak. The load was established during preliminary testing, although VO₂ measurements were conducted and evaluated during the trial, to ensure the estimated load elicited 55% VO₂ peak, and to account for a potential drift in VO₂ during exercise. In case of insufficient or excessive loading, adjustments were implemented. Sprint exercise trial (SPT). The SPT trial consisted of a 5-min warmup at 50 W, followed by three bouts of 30-s all-out sprint (Wingate tests) on an ergometer bike. Each Wingate test was interspersed by 4 min of active recovery on 5 W. Resistance exercise trial (RES). The RES trial was based on bilateral knee extension exercise. The trial was initiated by a warmup consisting of 3 sets of 10 repetitions with a load corresponding to 50% of the 10-RM load. Each of the warm-up sets were interspersed by 2 min of rest. Following the warm-up 6 sets of 10 repetitions were performed at a load corresponding to 10-RM with each set interspersed by 2 min of rest.

Statistics. All data was expressed as mean±SEM unless otherwise specified. A student's t-test was used for pair-wise comparisons. Unless otherwise specified, statistical significance was set as P<0.05.

### Results

Regular physical activity is a powerful intervention that can reduce obesity and confer protection against obesity-associated metabolic diseases (1-4). Conversely, physical inactivity increases the risk of developing obesity and type 2 diabetes (5, 6). The magnitude of these cardiometabolic benefits is comparable, and in some cases even greater, than first line pharmacological therapies such as metformin (7-9). The mechanisms responsible for the cardiometabolic benefits of exercise are incompletely understood but are likely to extend beyond activity-associated increases in energy expenditure alone.

One endocrine mechanism that underlies the metabolic benefits of physical activity is the exercise-inducible production of circulating signaling molecules. These secreted factors have been proposed to mediate tissue crosstalk and function as molecular transducers of the metabolic benefits of physical activity (10). Using candidate approaches, previous studies have identified several bioactive circulating metabolites that mediate salutary remodeling of adipose and muscle tissues following exercise training (11-14). However, untargeted strategies to systematically identify novel metabolites has been more limited.

Using untargeted metabolomics of blood plasma, here Lac-Phe (N-lactoyl-phenylalanine), a metabolite of previously unknown function, is identified as one of the most robustly induced circulating metabolites following physical activity in both mice and humans. Lac-Phe is produced in CNDP2+ cells including peripheral macrophages and epithelial cells. Gain- and loss-of-function studies establish that Lac-Phe suppresses adiposity and obesity by directly suppressing food intake. Lastly, sprint, endurance, and resistance exercise in humans all lead to dramatic and sustained elevations of Lac-Phe levels in blood plasma. It is therefore concluded that Lac-Phe is an exercise-inducible signaling metabolite that mediates the anti-obesity effects of physical activity.

### Acute Treadmill Running Robustly Increases Circulating Lac-Phe in Mice

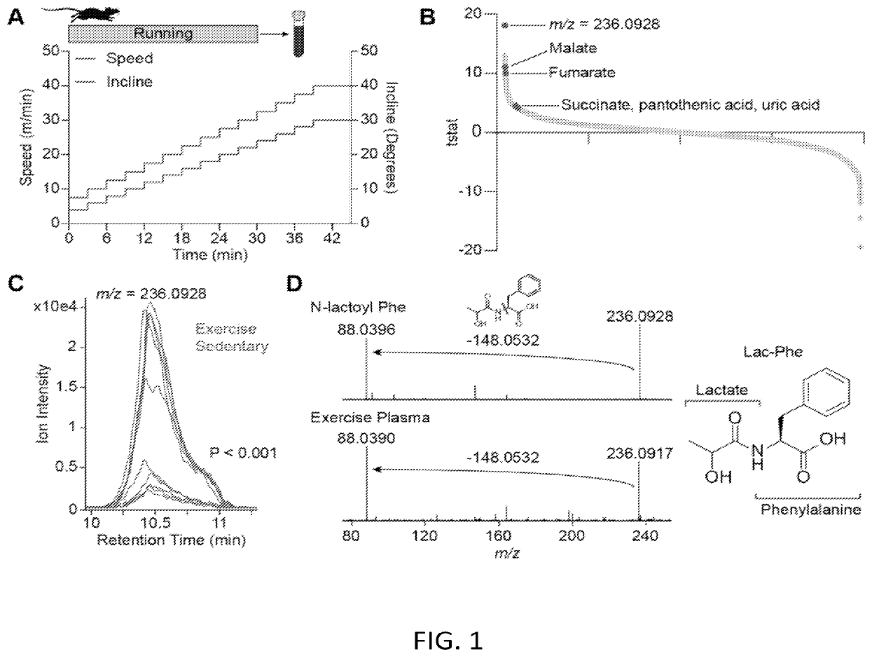

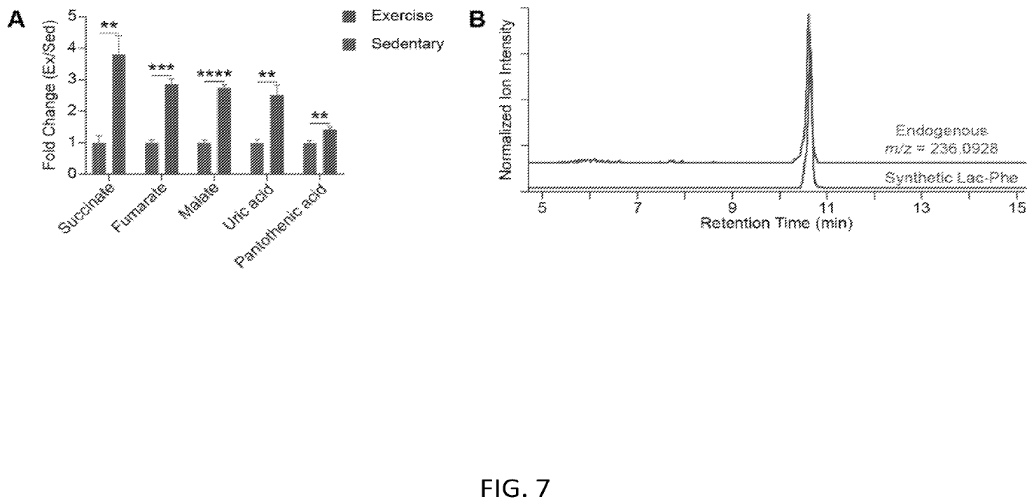

To measure exercise-induced circulating metabolites in a global and unbiased manner, untargeted metabolomic profiling by liquid chromatography-mass spectrometry (LC-MS) of acetonitrile/methanol-extracted blood plasma was performed in mice following an acute bout of treadmill running (FIG. 1 A). In this exercise protocol, the treadmill speed and incline were increased every three minutes until the mice reached exhaustion (see Methods). Importantly, the untargeted metabolomics pipeline does not require a priori knowledge of the chemical structures of the peaks detected, thereby enabling a broader and more unbiased sampling of the exercise-regulated changes in blood plasma. As expected, metabolites previously detected in targeted mass spectrometry experiments to be robustly increased by physical activity, including intermediates of the TCA cycle (e.g., succinate, fumarate, malate) as well as products of nucleotide catabolism (e.g. urate), were found in the metabolomics dataset to be increased by 2- to 4-fold in exercise relative to sedentary conditions (FIG. 1 B and FIG. 7 A). From the 7,752 peaks detected in this metabolomics experiment, the most statistically significant change was in an unknown peak of mass-to-charge ratio (m/z) of 236.0928 and retention time ˜10.5 min (FIG. 1 C). This metabolite was robustly elevated by >5-fold relative to control mice (P<0.001). The parent mass of 236.0928 was consistent with a molecular formula of [C₁₂H₁₄NO₄]⁻ but such a molecule did not match any obvious metabolite in the existing databases.

To determine the chemical identity of this exercise-induced metabolite, tandem mass spectrometry experiments were performed and a prominent daughter ion of m/z=88.040 was observed. This daughter peak and the mass loss (148.0532) matched molecular formulas of [C₃H₆NO₂]⁻ and [C₉H₈O₂], respectively (FIG. 1 D). From these fragmentation spectra, the parent metabolite was tentatively assigned to N-lactoyl-phenylalanine, an amidated conjugate of lactate and phenylalanine (“Lac-Phe”, FIG. 1 D). An authentic Lac-Phe standard produced via chemical synthesis exhibited identical MS/MS spectra (FIG. 1 D) and retention times (FIG. 7 B) as the endogenous m/z=236.0928 peak, thereby confirming the structural assignment. Absolute quantitation of Lac-Phe levels revealed circulating basal and exercise-induced concentrations of 0.5±0.4 μM and 2.7±0.9 μM (mean±SEM), respectively. These data establish Lac-Phe as one of the most robustly induced plasma metabolites following acute treadmill running in mice.

### Lac-Phe Suppresses Food Intake and Obesity

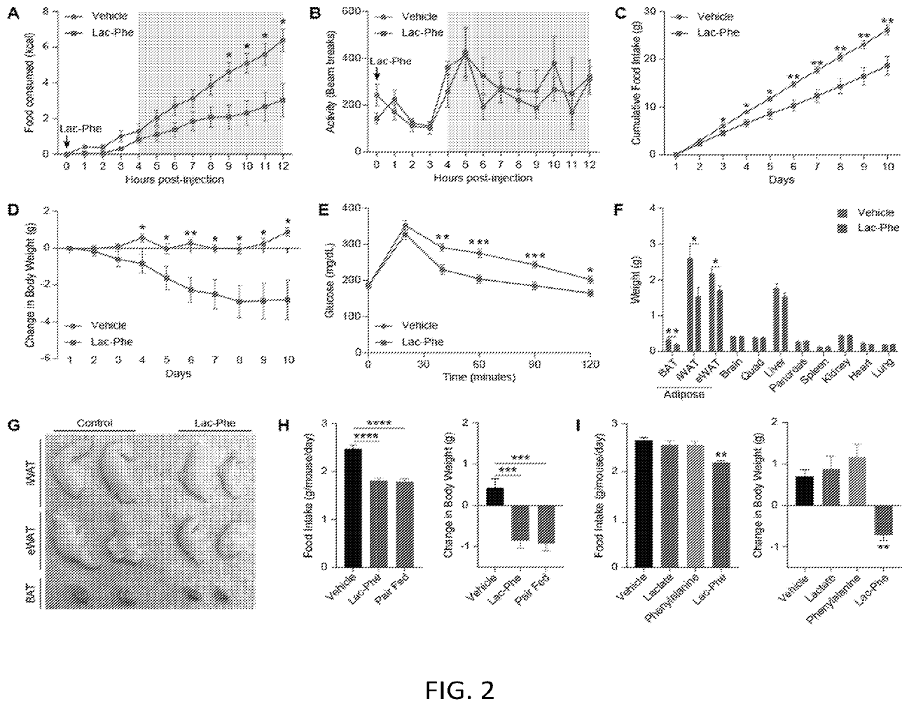

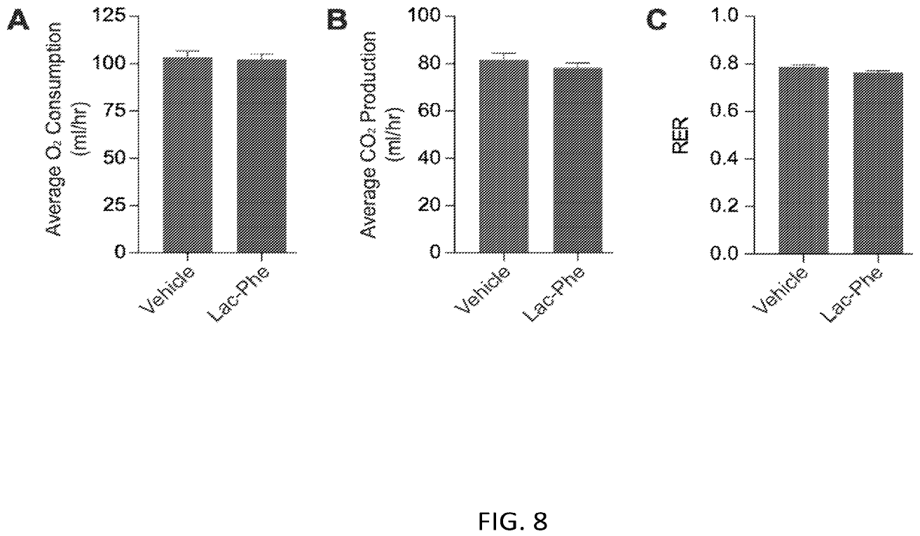

Lac-Phe is a poorly studied metabolite of unknown function. To determine if Lac-Phe might have a functional role in metabolic homeostasis, metabolic chambers were used to determine the effect of acute Lac-Phe administration (50 mg/kg, intraperitoneal [IP]) to diet-induced obese (DIO) mice. Importantly, these experiments were performed at a dose of Lac-Phe such that peak plasma concentrations were comparable to that observed after a single bout of exercise training (plasma levels 30 min post injection, mean±SEM, 3.8 μM±1.9 μM). As shown in FIG. 2 A, acute Lac-Phe administration suppressed food intake by 52% compared to vehicle-treated mice over a 12 h post-administration period (mean±SEM, vehicle 6.4±0.6 kcal/mouse; Lac-Phe 3.0±1.0 kcal/mouse). Importantly, ambulatory activity was not different between groups (FIG. 2 B), demonstrating that the suppression of food intake was not simply due to reduction in overall movement. Acute Lac-Phe treatment did not alter oxygen consumption (VO₂), carbon dioxide production (VCO₂), or respiratory exchange ratio (RER, FIG. 8 A-C). These data demonstrate that acute Lac-Phe administration to mice specifically suppresses energy intake without altering energy expenditure pathways.

The ability for Lac-Phe to chronically reduce food intake would be expected to reduce body weight and improve glucose and lipid homeostasis. To directly test this hypothesis, Lac-Phe was chronically administered to DIO mice (50 mg/kg/day, intraperitoneal [IP], once dose per day, see Methods) and changes in food intake and body weight were monitored over a 10-day period. As expected, chronic Lac-Phe treated animals exhibited reduced cumulative food intake compared to control mice (mean±SEM, Lac-Phe 2.1±0.1 g/mouse/day; vehicle 2.9±0.1 g/mouse/day, FIG. 2 C). This decrease in food intake in Lac-Phe treated mice was associated with a concomitant reduction in body weight (mean±SEM, −2.8±1.1 g, corresponding to −7% change in total body weight, FIG. 2 D). On day 10, a glucose tolerance test was performed, which showed improved glucose clearance in DIO mice treated with Lac-Phe versus vehicle (FIG. 2 E). Dissection of tissues from Lac-Phe-treated mice revealed significant reductions in adipose tissue mass, including smaller epididymal fat (eWAT, −20%), subcutaneous inguinal fat (iWAT, −41%), and brown fat (BAT, −41%) depots compared to vehicle-treated mice (FIG. 2 F, G). Total lean mass, as estimated by the weights of the other organs harvested, was unaffected (FIG. 2 F).

Next, a pair feeding experiment was performed in which food was restricted to a pair-fed group at levels identical to that consumed by Lac-Phe treated mice (mean±SEM, pair-fed 1.8±0.1 g/mouse/day; control 2.5±0.1 g/mouse/day, corresponding to a 28% reduction of food, FIG. 2 H). Under these conditions, control pair-fed mice exhibited identical changes to body weight as mice treated with Lac-Phe (FIG. 2 H), confirming that the effects of Lac-Phe on adiposity and obesity are due to suppression of food intake and not due to any changes in activity or metabolic rate.

Lastly, the structural features of Lac-Phe that are required for its anorexigenic and anti-obesity effects were explored. Notably, Lac-Phe is composed of two “halves,” lactate and phenylalanine. Identical experiments to those described above were performed to directly compare Lac-Phe with lactate and phenylalanine (50 mg/kg, IP daily). Lac-Phe once again suppressed food intake and body weight compared to control mice (FIG. 2 I). By contrast, lactate or phenylalanine administration at equivalent doses were identical to vehicle treated mice in both food intake and body weight (FIG. 2 I). These data establish that elevation of Lac-Phe in diet-induced obese mice at a dose that mimics a single bout of physical activity suppresses food intake and reduces obesity in a manner that requires the intact amidated conjugate.

CNDP2- and Lactate-Dependent Lac-Phe Secretion from Macrophages

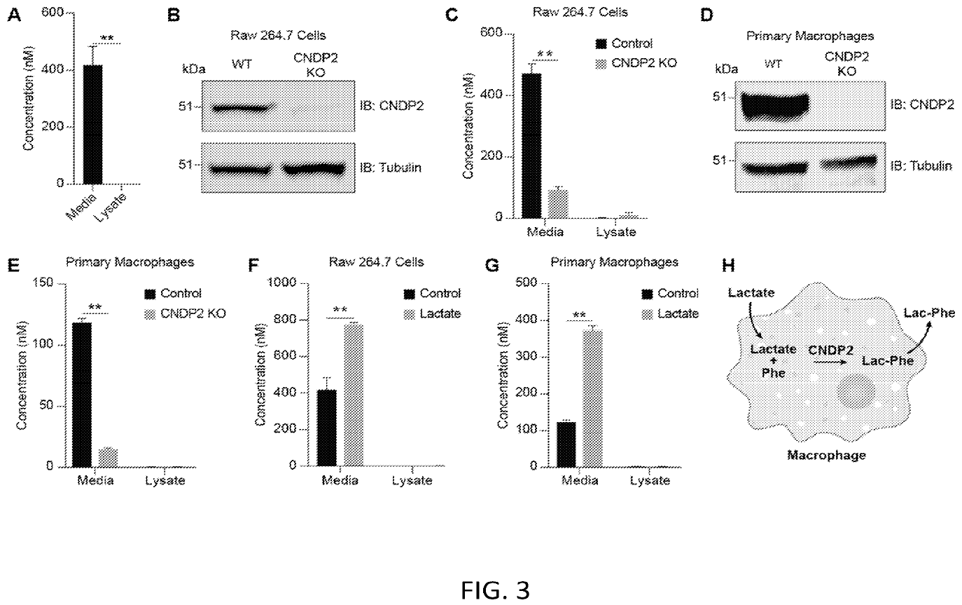

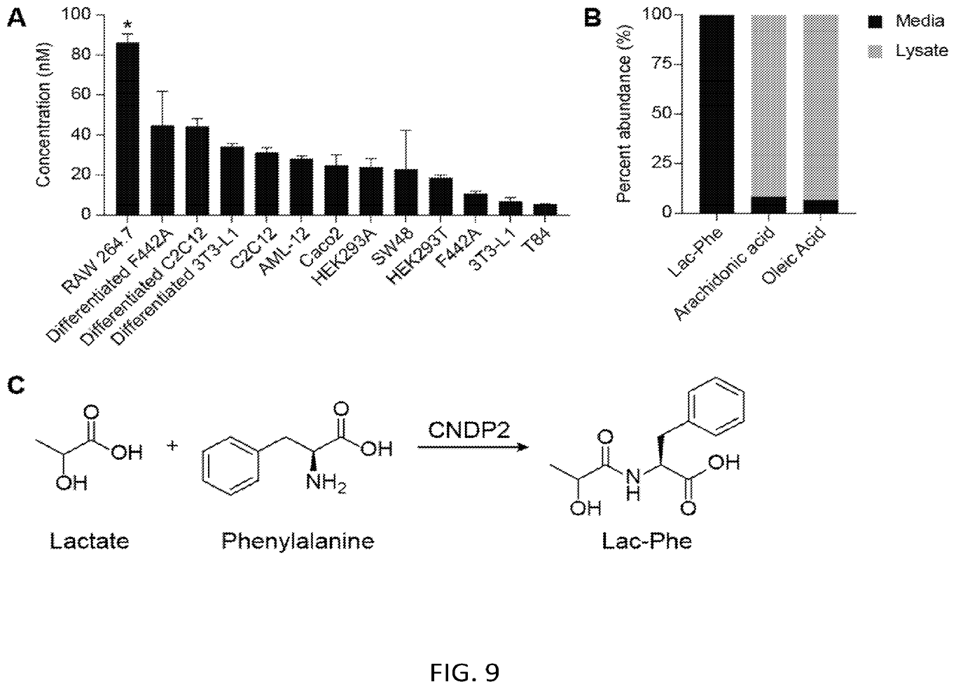

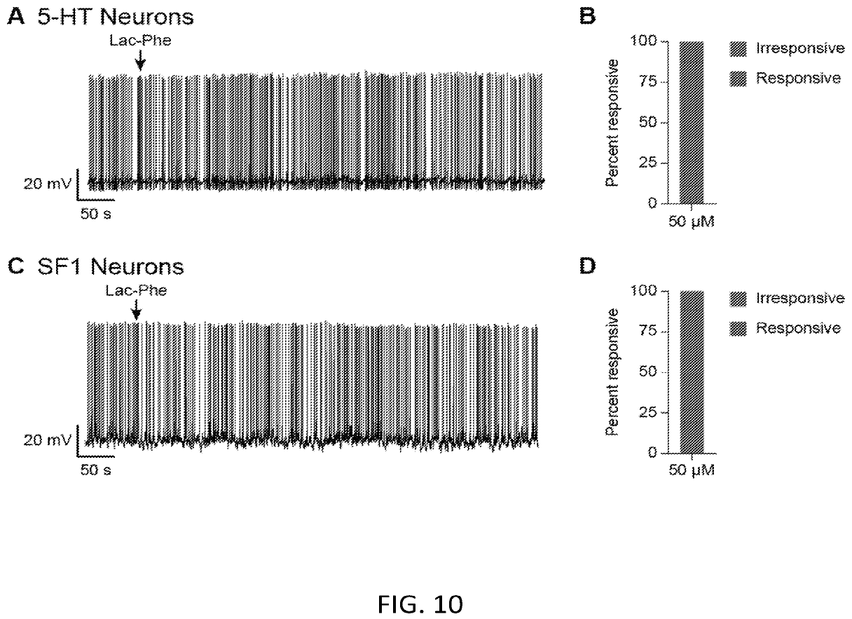

To better understand how Lac-Phe is produced, an aim was first to identify a cell line that exhibited robust Lac-Phe biosynthesis and secretion in vitro. Towards this end, levels of secreted Lac-Phe in conditioned media were measured by LC-MS/MS across a panel of 13 cell lines. The murine macrophage cell line RAW264.7 was identified to robustly secrete Lac-Phe into conditioned media (FIG. 9 A). Nearly all of the Lac-Phe was secreted from RAW264.7 cells; <1% was found intracellularly (FIG. 3 A). By contrast, levels of other intracellular lipids, such as arachidonic acid and oleic acid, were found as expected to be enriched in the cell lysate (FIG. 9 B). The other 12 cell lines examined exhibited a wide range of Lac-Phe levels in conditioned media that were ˜50-90% lower than the level observed from RAW264.7 cells (FIG. 9 A). These data therefore establish robust secretion of Lac-Phe from cells in culture and furthermore identify RAW264.7 macrophages as a model cell line for studying this process.

Previously, a cytosolic enzyme called CNDP2 has been shown to catalyze the condensation of lactate and phenylalanine to generate Lac-Phe in vitro (FIG. 9 C) (15). However, the physiologic relevance as well as the directionality of this CNDP2-catalyzed reaction has not been previously evaluated in more complex cellular and organismal settings. CNDP2-KO RAW264.7 cells were generated with CRISPR/Cas9 and loss of CNDP2 protein was validated using an anti-CNDP2 antibody (FIG. 3 B). As shown in FIG. 3 C, extracellular Lac-Phe levels were reduced by >75% in CNDP2-KO relative to control cells. Primary peritoneal macrophages were also obtained from CNDP2-KO and control WT mice (FIG. 3 D). Once again, extracellular Lac-Phe levels were >85% reduced in primary CNDP2-KO macrophages versus WT control macrophages (FIG. 3 E). Since lactate is both increased by exercise and could function as a metabolic precursor for Lac-Phe, whether extracellular lactate could stimulate Lac-Phe production through a mass action-type mechanism was tested. Addition of lactate to the conditioned media at concentrations reached during intense exercise (25 mM) increased extracellular levels of Lac-Phe by +85% in RAW264.7 cells (FIG. 3 F) and +200% in primary macrophages (FIG. 3 G). Taken together, these data therefore establish CNDP2 as the principal biosynthetic enzyme for Lac-Phe production in cell culture. These data also identify muscle-derived lactate as a candidate metabolic precursor that drives Lac-Phe production during acute exercise (FIG. 3 H).

### Genetic Ablation of Lac-Phe Biosynthesis Confers Resistance to the Anti-Obesity Effects of Exercise Training

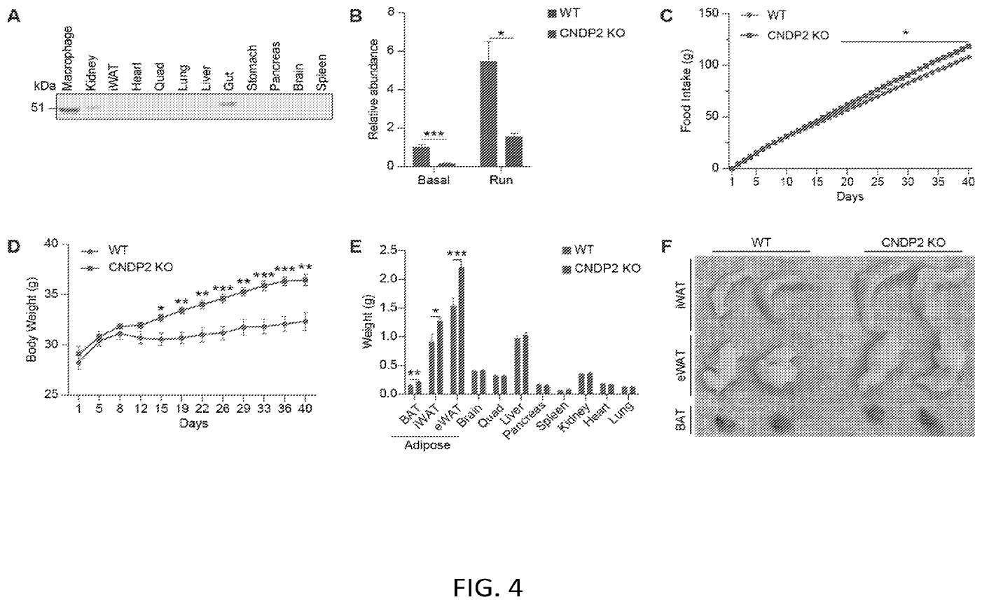

Next, CNDP2-KO mice were used as a model for genetic ablation of Lac-Phe. First, an anti-CNDP2 antibody was used to determine the tissue expression of CNDP2 protein in mice. As expected, peritoneal macrophages exhibited the highest CNDP2 expression. CNDP2 protein expression was also detected at lower levels in kidney and gut (FIG. 4 A). These protein-level data were consistent with publicly available microarray data showing enrichment of Cndp2 expression in macrophages, kidney, and intestine (BioGPS) (16) as well as publicly available single-cell data showing enrichment of Cndp2 expression in monocytes and macrophages (Tabula Muris) (17). Plasma levels of Lac-Phe in WT and CNDP2-KO mice were next measured under both basal and post-treadmill run conditions. Circulating Lac-Phe was dramatically reduced by >85% and >70% in basal and post-exercise conditions, respectively, in CNDP2-KO mice relative to WT control animals (FIG. 4 B). These data indicate that CNDP2 is a principal biosynthetic enzyme for Lac-Phe in vivo. The residual increase in Lac-Phe with exercise likely reflects additional, CNDP2-independent pathways for Lac-Phe biosynthesis.

WT and CNDP2-KO mice were next evaluated for food intake and obesity phenotypes. Because Lac-Phe is induced by exercise training, WT and CNDP2-KO mice were subjected to high fat diet (60% kcal from fat) in combination with a chronic treadmill running regimen (see Methods). Body weights at the beginning of this experiment were not different between WT and CNDP2-KO mice (mean±SEM, WT, 29.1±0.8 g; CNDP2-KO, 28.2±0.7 g; P>0.05). However, starting from day 10 on the training/obesogenic diet protocol, CNDP2-KO mice began to exhibit increased food intake compared to control mice (FIG. 4 C). This increased feeding behavior persisted until the end of the experiment on day 40. Consistent with this feeding behavior, CNDP2-KO mice also exhibited increasingly divergent body weight compared to control mice, with final body weight in CNDP2-KO mice+13% higher versus WT mice (mean±SEM, WT, 32.3±0.9 g; CNDP2-KO, 36.4±0.6 g, FIG. 4 D). Importantly, total run times in this experiment were equivalent between groups (mean±SEM, WT 41±1.2 min/day; KO 41±1.7 min/day). Lastly, dissection of tissues revealed an increase in adipose tissue mass in CNDP2-KO compared to control, whereas lean mass was similar (FIG. 4 E, F). From these data it is concluded that ablation of Lac-Phe leads to increased energy intake and weight gain during exercise training.

### Lac-Phe Directly Acts on Orexigenic AgRP and Anorexigenic POMC Neurons

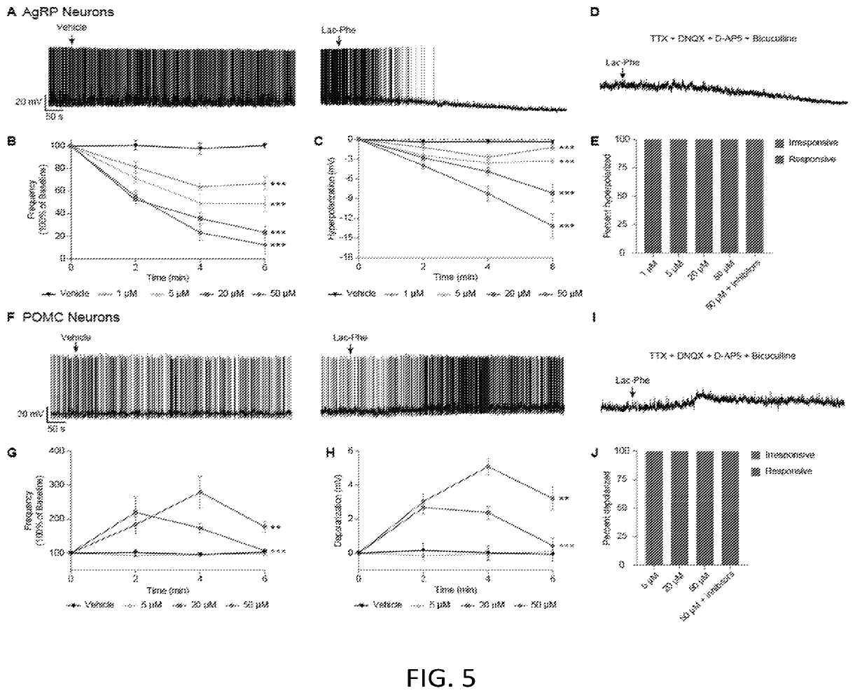

The brain plays a fundamental role in the regulation of appetite and body weight. Therefore, whole-cell patch-clamp slice electrophysiology was used to determine the effect of Lac-Phe directly on a number of neuronal populations known to regulate food intake. The focus was first on the Agouti-related peptide (AgRP) and proopiomelanocortin (POMC)-expressing neurons. These two neuronal populations are located in the arcuate nucleus of the hypothalamus (ARH) and have well-established roles as central orexigenic and anorexigenic drivers, respectively, of feeding behavior (18-24). Representative electrophysiology recordings for AgRP neurons are shown in FIG. 5 A. Consistent with its appetite suppressive effects in vivo, Lac-Phe (50 μM) rapidly inhibited orexigenic AgRP neurons, as demonstrated by decreased firing frequency rate and hyperpolarization of the resting membrane potential (FIG. 5 A). The inhibitory action of Lac-Phe was dose responsive, with inhibitory activity at concentrations as low as 1 μM which is similar to physiologic levels in circulation (FIG. 5 B, C). In addition, the hyperpolarization induced by Lac-Phe persisted in the presence of TTX (a sodium channel blocker), DNQX (the AMPA glutamatergic receptor antagonist), D-AP5 (the NMDA glutamatergic receptor antagonist), and bicuculline (the GABA_{A} receptor antagonist) (FIG. 5 D). Quantification revealed that >70% of neurons were hyperpolarized in response to all concentrations of Lac-Phe tested under either baseline or inhibitor-treated conditions (P>0.05 for the effect of inhibitor treatment, FIG. 5 E). These results indicate that the inhibitory effects of Lac-Phe on AgRP neurons are mediated via a direct action rather than through a circuitry or pre-synaptic mechanism.

The effects of Lac-Phe on anorexigenic POMC+ neurons were also examined. Lac-Phe could rapidly activate POMC neurons as demonstrated by increased firing frequency and depolarization (FIG. 5 F). Notably, the effects of Lac-Phe to activate POMC neurons was less potent than Lac-Phe inhibition of AgRP neurons, requiring a concentration of at least 20 μM (FIG. 5 G, H). The depolarization induced by Lac-Phe persisted in the presence of the cocktail of inhibitors mentioned previously, indicating that the stimulatory effects of Lac-Phe on POMC neurons are also mediated via a direct action (FIG. 5 I, J).

Lastly, these slice electrophysiology studies were expanded to explore the effects of Lac-Phe in two other neural populations with potential relevance for regulation of feeding (FIG. 10 A-D). No effect of Lac-Phe on the excitability of serotonin (5-HT)-positive neurons in the dorsal raphe nuclei (DRN) or on seroidogenic factor 1 (SF1)-positive neurons of the ventromedial hypothalamic nucleus (VMH) was observed, indicating that Lac-Phe acts in a cell type-specific manner. Taken together, it can be concluded Lac-Phe can directly act to inhibit orexigenic AgRP neurons and stimulate anorexigenic POMC neurons in brain slices.

### Robust and Sustained Elevations of Circulating Lac-Phe Following Exercise in Humans

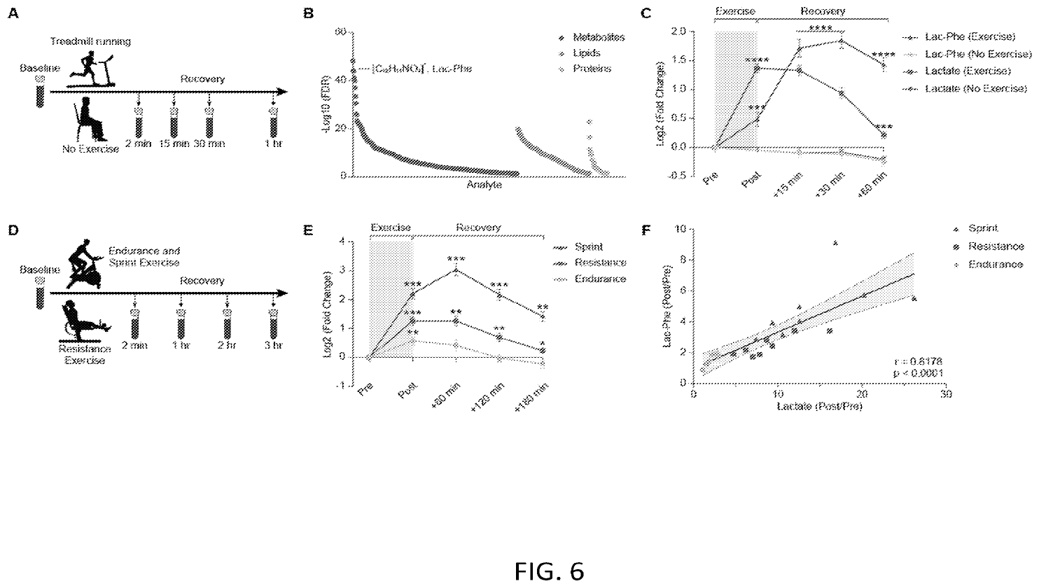

To determine whether circulating Lac-Phe also exhibited exercise-inducible elevation in humans, Lac-Phe levels were measured in two independent human exercise cohorts. First, untargeted plasma mass spectrometry data from a previously published deeply phenotyped acute treadmill running exercise cohort was re-analyzed (FIG. 6 A) (25). These individuals (N=36) were fasted overnight and then subjected to symptom-limited cardiopulmonary exercise testing by treadmill running. This cohort was 58% male, of ages between 40-75 years old, with an average BMI of 28.4±0.9 kg/m² (mean±SEM). Steady-state plasma glucose (SSPG) as determined by the modified insulin suppression test was determined to be 153±67 mg/dl (mean±SEM). The majority of the participants (86%) exceeded a respiratory exchange ratio (RER)>1.05 at peak exercise while the remaining individuals reached heart rates of >95% of their age-adjusted maximum predicted heart rate. All the plasma samples from this cohort had been previously subjected to in-depth multi-omic profiling (proteomics, metabolomics, transcriptomics).

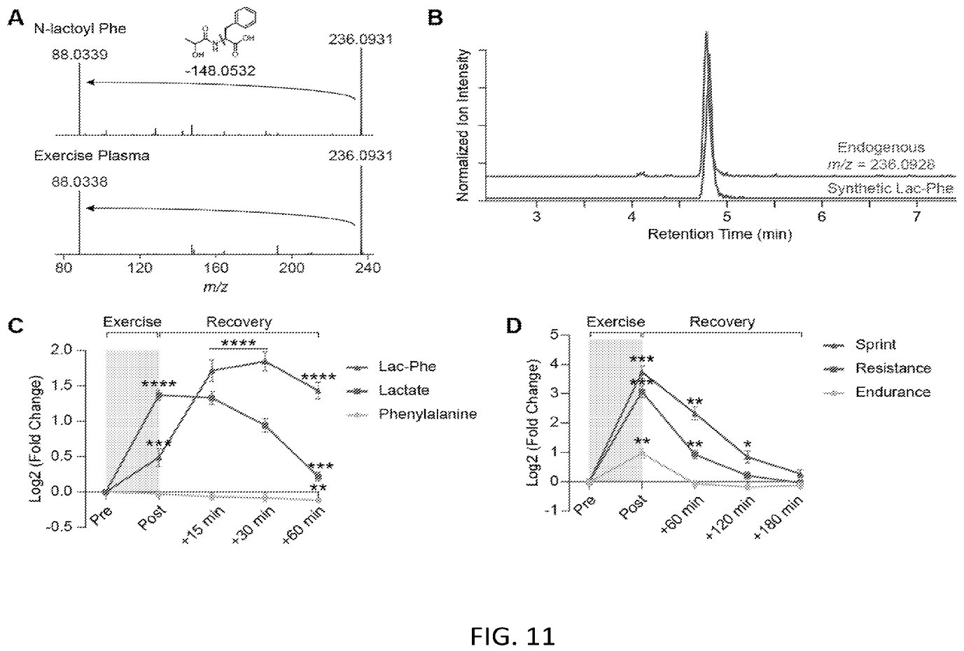

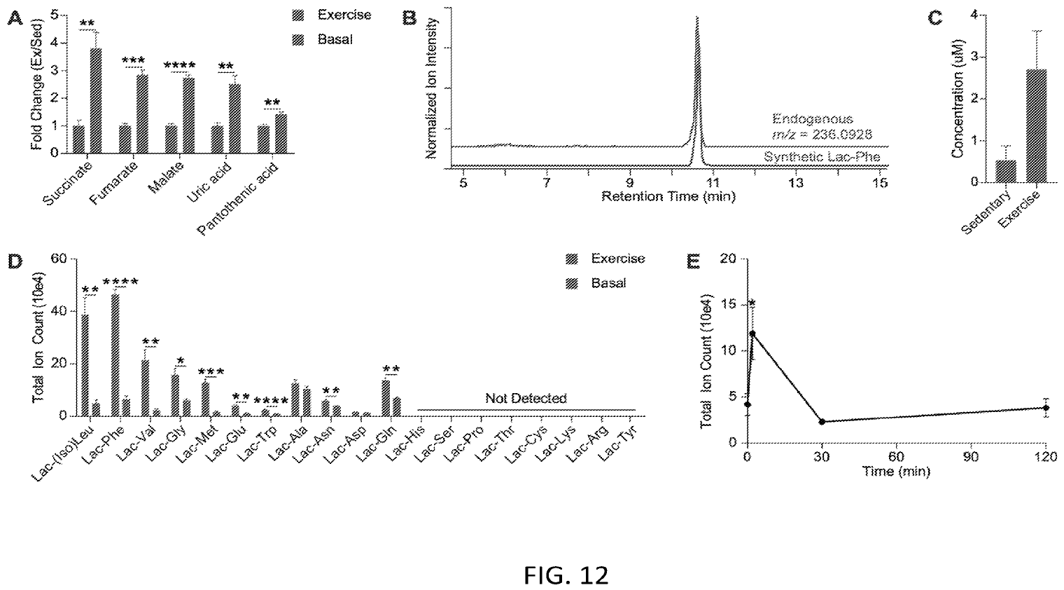

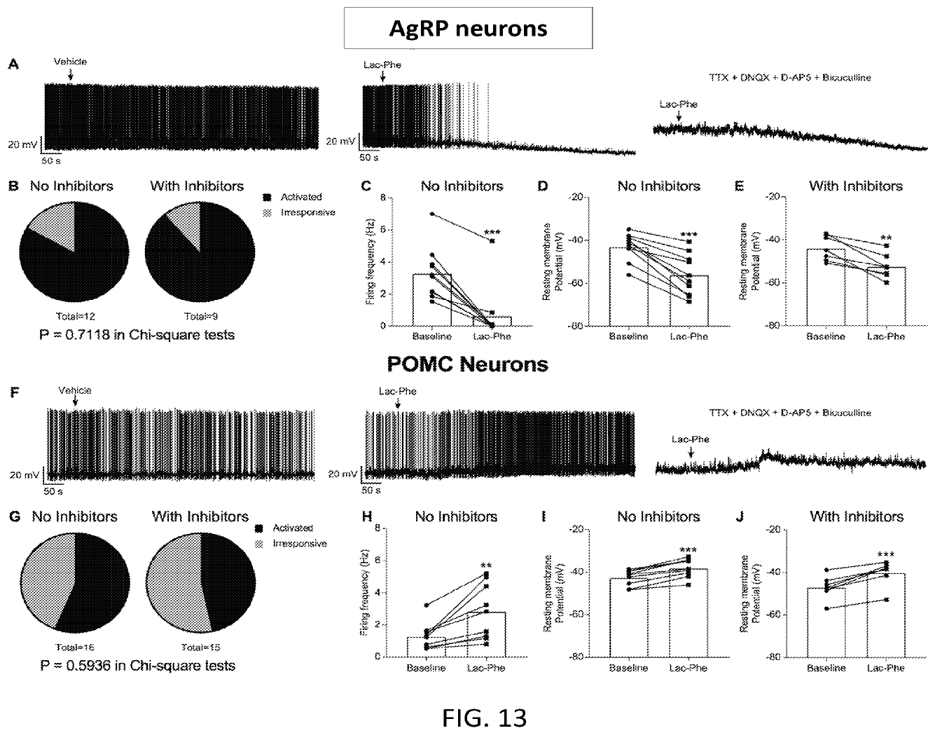

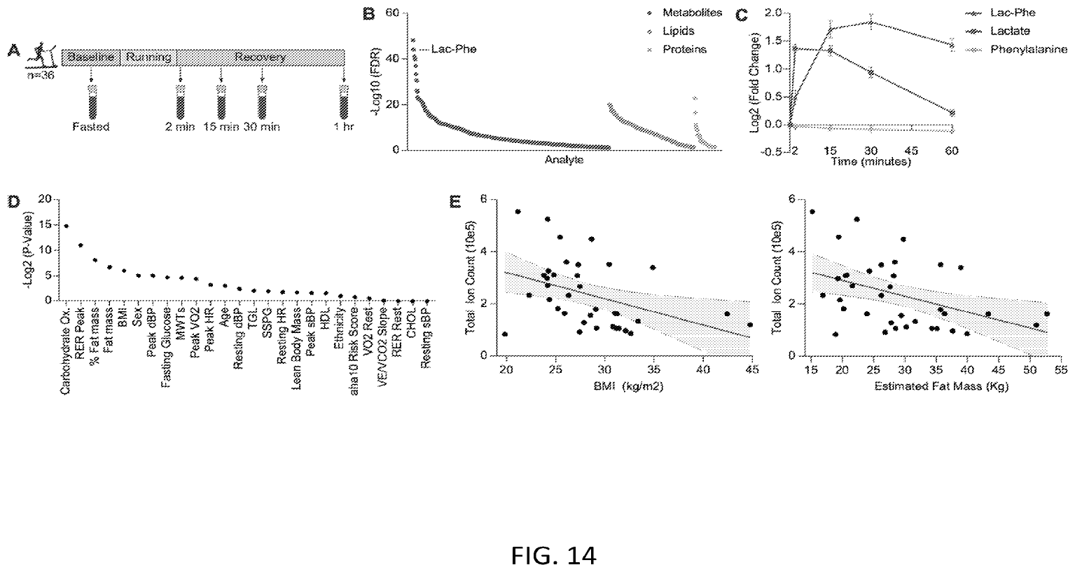

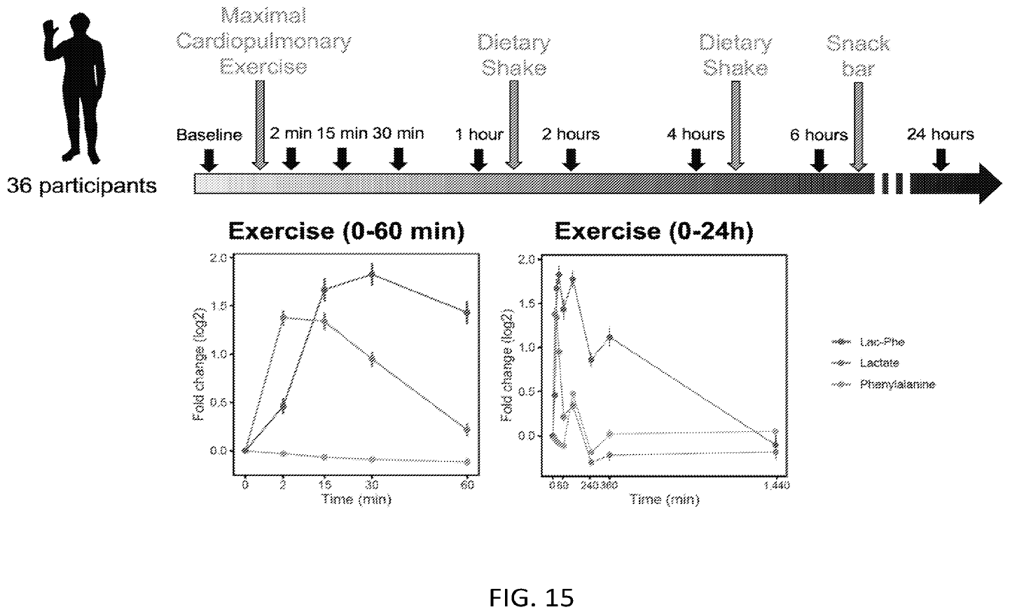

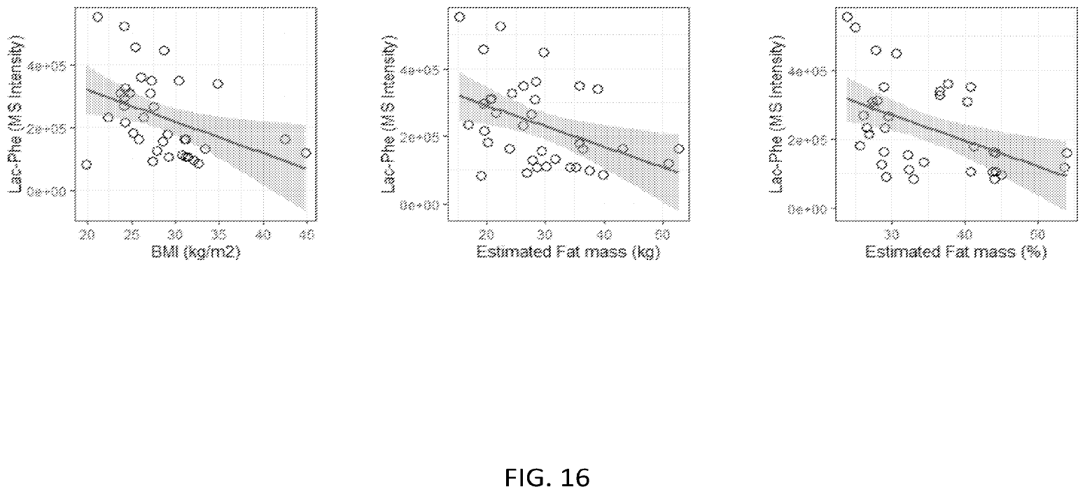

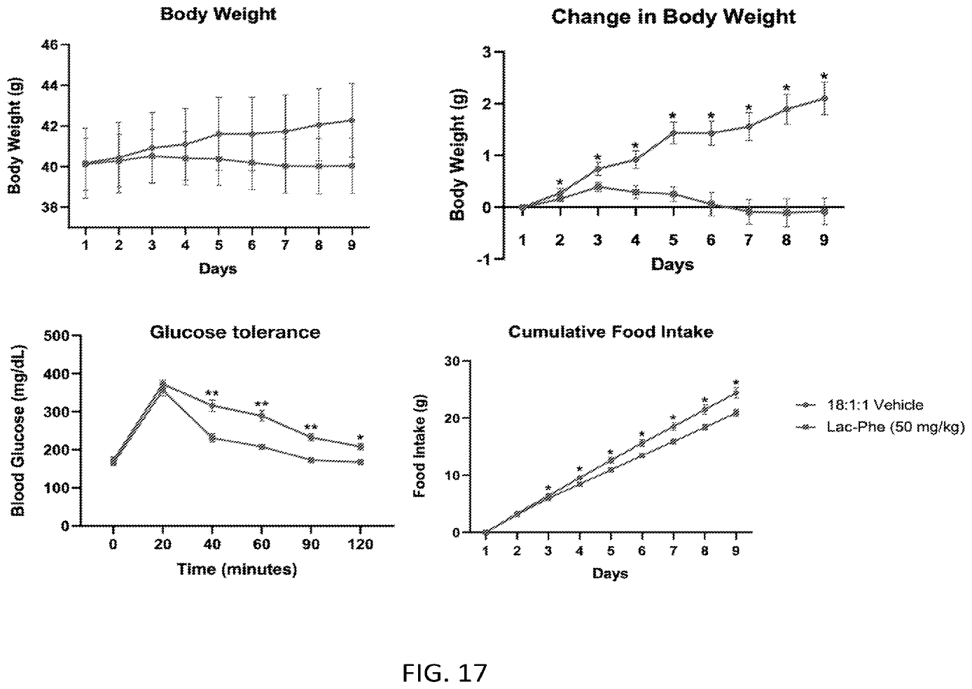

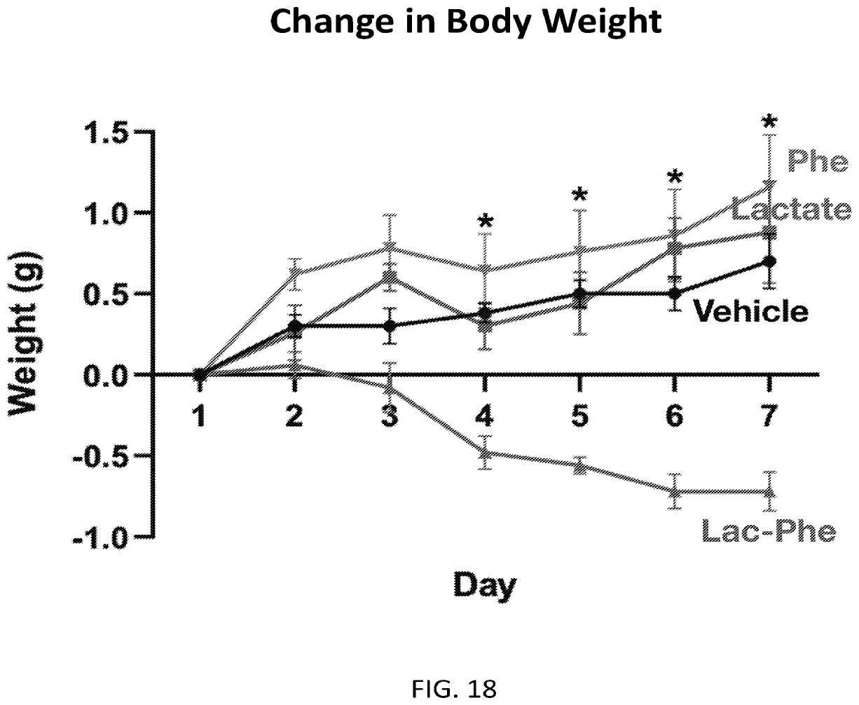

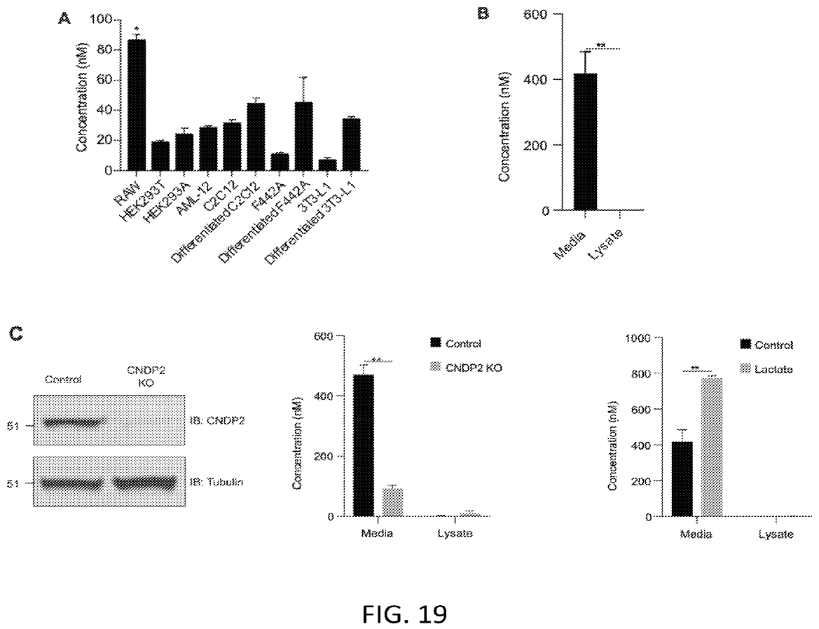

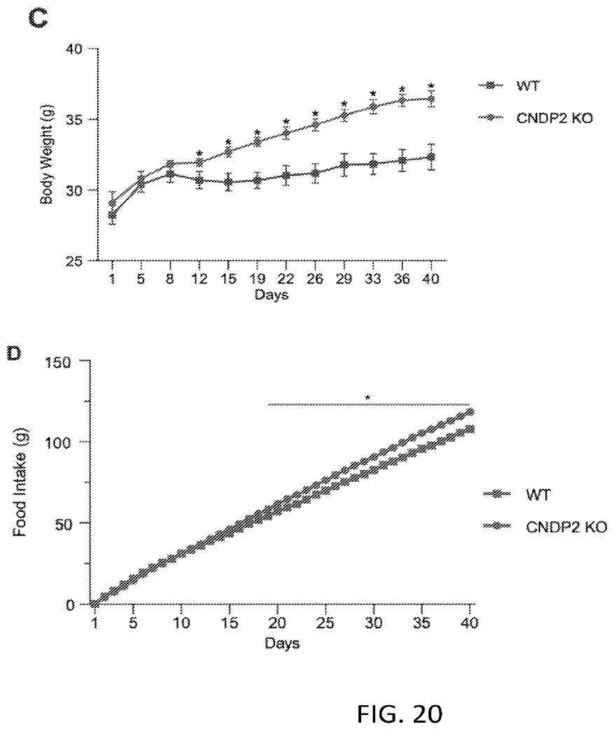

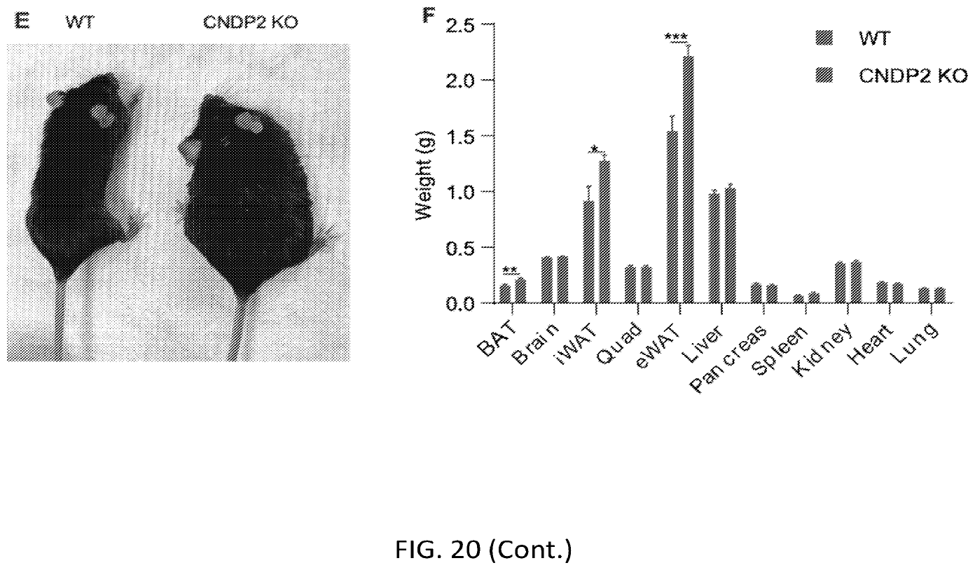

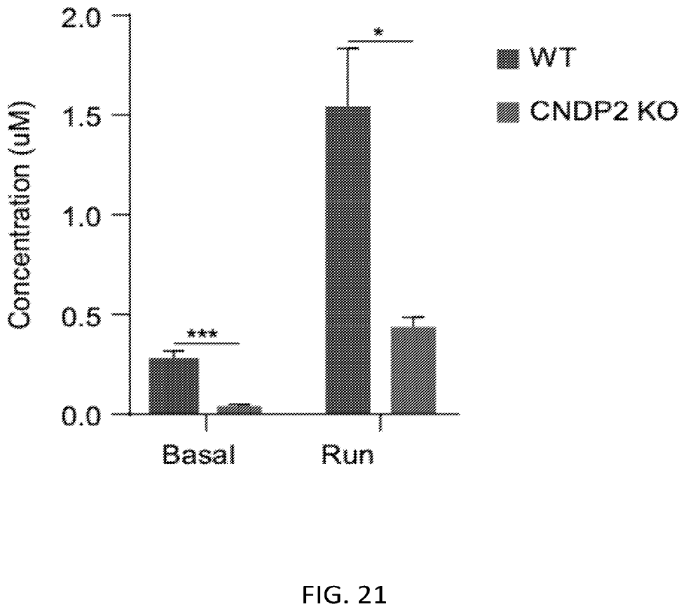

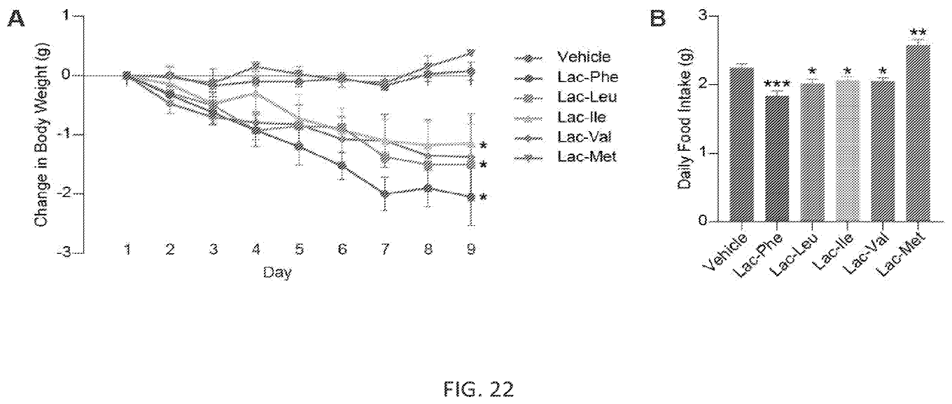

Remarkably, out of the 1,807 analytes that were classified as small molecule metabolites, lipids, or proteins, a yet unassigned peak corresponding to a metabolite with chemical formula matching Lac-Phe (C₁₂H₁₄NO₄⁻) was ranked third most significantly induced by exercise in the entire dataset (FIG. 6 B). Tandem mass spectrometry experiments revealed a fragmentation spectrum with the characteristic m/z=88 daughter ion (FIG. 11 A) and identical retention times in comparison to an authentic Lac-Phe standard (FIG. 11 B). It is therefore concluded that this previously unassigned, exercise-induced metabolite in humans is Lac-Phe. With this structural assignment in hand, it was found that plasma Lac-Phe levels showed a robust and sustained exercise-induced elevation which peaked at 4-fold over baseline at 30 min post-exercise and persisted beyond 1 h-post exercise (FIG. 6 C). In comparison, lactate levels peaked at the cession of exercise and quickly returned to baseline by 1 h. Plasma phenylalanine levels were largely unchanged throughout the exercise intervention (FIG. 11 C). In a control cohort of individuals that did not undergo treadmill exercise, Lac-Phe was in fact slightly decreased over the same time course, thereby establishing the exercise-dependent accumulation of Lac-Phe in humans (FIG. 6 C). From these data, it is concluded that Lac-Phe is also one of the most robustly increased metabolites in human plasma following an acute bout of treadmill running.

To determine the generality of Lac-Phe elevation following human exercise, plasma Lac-Phe levels in a second human exercise cohort consisting of individuals that had each been tested in three distinct exercise trials were measured (endurance, sprint, and resistance, see Methods, FIG. 6 D). Eight healthy young men aged 28.0±1.3 yrs (mean±SEM) and average BMI 23.5±0.7 kg/m² (mean±SEM) were recruited into this study. The individuals were fasted prior to the exercise trials, which were performed in randomized order and separated by at least 10 days. The endurance exercise trial consisted of 90 min continuous cycling at ˜60% peak VO₂; the sprint exercise trial consisted of three bouts of 30 sec all out sprint (Wingate test) on an ergometer bike; and the resistance exercise trial consisted of bilateral knee extension (6 sets of 10 repetitions at a load corresponding to 10-RM). Blood plasma was collected prior to exercise and at several time points post-exercise, and Lac-Phe was measured from acetonitrile/methanol-extracted plasma by LC-MS/MS. Lac-Phe once again exhibited robust and sustained elevations across all three exercise modalities (FIG. 6 E). Sprint exercise exhibited the most dramatic accumulation of plasma Lac-Phe (peaking at 8-fold elevation at 1 h post exercise), followed by resistance training and then endurance training. Because the magnitude of Lac-Phe elevation across exercise modalities matched that of lactate (sprint>resistance>endurance, FIG. 11 D), the relationship between Lac-Phe and lactate levels was examined. As shown in FIG. 6 F, the pre- versus immediately post-exercise fold-change of Lac-Phe and lactate exhibited a strong and nearly linear correlation (Pearson correlation coefficient r=0.82, P<0.001). These data establish that distinct physical activity modalities lead to robust and sustained elevation of circulating Lac-Phe levels in humans.

### DISCUSSION

Here an exercise-inducible anti-obesity signaling molecule mediated by a circulating lactate-derived metabolite called Lac-Phe is identified. The pharmacological gain- and genetic loss-of-function experiments in mice, as well as cellular studies in culture, provide three lines of evidence that Lac-Phe is an important circulating molecule for the anti-obesity effects of physical activity: (1) elevation of Lac-Phe to levels achieved during acute exercise are sufficient to reduce food intake and obesity; (2) genetic ablation of Lac-Phe biosynthesis in mice confers resistance to the anti-obesity effects of exercise training; and (3) Lac-Phe directly regulates the excitability of neurons previously established to regulate feeding behaviors in vitro. Further supporting these findings, Lac-Phe is identified as one of the most robustly induced molecular changes in blood plasma following diverse physical activity modalities in humans.

Strenuous exercise has been well-documented to acutely suppress food intake in mice, a behavioral phenotype that correlates with post-exercise suppression and stimulation of neuronal activity in AgRP and POMC neurons, respectively (26-28). While specific candidate molecules, including ghrelin, have been proposed to contribute to these effects, the role of additional exercise-inducible signaling molecules on these food intake pathways has remained poorly understood. The data designate Lac-Phe as an exercise-inducible molecule that functions upstream of these critical central pathways that regulate feeding behaviors. In humans, the effects of exercise on appetite and food intake are more complex, depending on study cohort and the type, duration, or intensity of physical activity (29-31). These human data could be interpreted to suggest that exercise may also induce production of additional, as of yet unknown appetite-regulating molecules besides Lac-Phe.

As a circulating regulator of obesity and food intake, Lac-Phe exhibits both similarities and also important differences compared to other peripherally-derived appetite-regulating hormones. Lac-Phe inhibits feeding behaviors just like many other peptide hormones including GLP-1, GDF15, CCK, PYY, and leptin. Both Lac-Phe and GDF15 are produced and secreted from similar cell types and anatomical locations, including macrophages, the intestine, and the kidney (32, 33). However, there are also important differences. For instance, the dramatic, robust, and sustained exercise-inducible elevation of Lac-Phe appears to be unique to this molecule, in contrast to the other peripherally-derived peptide hormones which are predominantly regulated by nutrients and only modestly changed, if at all, by physical activity (34-36). Furthermore, Lac-Phe is a metabolite (MW ˜250 Da), whereas the other appetite hormones are much larger polypeptides (e.g., GLP-1, MW ˜3000 Da) or proteins (e.g., GDF15, MW ˜15 kDa).

The present studies of Lac-Phe production and regulation also provide surprising insights about the cellular and biochemical mechanisms underlying the physiologic responses to physical activity. First, myocytes and muscle tissues have been a historical focus as a source for circulating molecular transducers of physical activity (37-39). That macrophages and other CNDP2+ cell types produce and secrete Lac-Phe suggests that many additional cell types can sense and respond to physical activity (13, 40). A potential common aspect of these other exercise-responsive cell types might be their ability to directly detect or otherwise import muscle-derived lactate. Second, that lactate functions as a metabolic precursor for Lac-Phe biosynthesis provides both a circuit as well as a biochemical logic for the robust elevation of Lac-Phe during exercise: namely, that exercise induces lactate secretion from contracting muscles, which is rapidly imported into CNDP2+ cell types and then re-exported back into the circulation in the amidated Lac-Phe form. Alternative metabolic derivatizations of lactate could also in principle constitute yet additional undiscovered exercise-inducible signaling molecules. Third, the observation that Lac-Phe exhibits robust and sustained elevation even at time points when lactate has returned back to baseline levels suggests that biochemical derivatization of lactate functions as an organismal second messenger system in which lactate sensing is coupled with long-lasting endocrine signaling. The sustained elevation of circulating Lac-Phe may in part reflect the fact that Lac-Phe, unlike lactate, cannot be directly re-incorporated back into pathways of primary metabolism.

Lastly, the benefits of physical activity extend beyond metabolic health alone. In the brain, exercise enhances mood, reduces anxiety and depression, and protects against dementia and other neurodegenerative diseases (41-43). Lac-Phe, by exhibiting direct action on neurons, represents an attractive candidate factor for mediating some of these additional benefits. Pharmacological targeting of the Lac-Phe pathway may therefore be useful for the treatment of obesity and these other disorders of the nervous system.

### REFERENCES

1. K. F. Eriksson, F. Lindgärde, Prevention of Type 2 (non-insulin-dependent) diabetes mellitus by diet and physical exercise. Diabetologia. 34, 891-898 (1991).
2. X.-R. Pan, G.-W. Li, J.-X. Wang, W.-Y. Yang, Z.-X. An, Z.-X. Hu, Juan-Lin, J.-Z. Xiao, H.-B. Cao, P.-A. Liu, X.-G. Jiang, Y.-Y. Jiang, J.-P. Wang, H. Zheng, H. Zhang, P. H. Bennett, B. V. Howard, Effects of diet and exercise in preventing NIDDM in people with impaired glucose tolerance. The Da Qing IGT and Diabetes Study. Diabetes Care. 20, 537-544 (1997).
3. W. J. Rejeski, E. H. Ip, A. G. Bertoni, G. A. Bray, G. Evans, E. W. Gregg, Q. Zhang, Lifestyle Change and Mobility in Obese Adults with Type 2 Diabetes. N. Engl. J. Med. 366, 1209-1217 (2012).
4. M. J. Stampfer, F. B. Hu, J. E. Manson, E. B. Rimm, W. C. Willett, Primary prevention of coronary heart disease in women through diet and lifestyle. N. Engl. J. Med. 343, 16-22 (2000).
5. S. P. Helmrich, D. R. Ragland, R. W. Leung, R. S. Paffenbarger, Physical activity and reduced occurrence of non-insulin-dependent diabetes mellitus. New English J. Med. 325, 147-152 (1991).
6. A. Rawshani, A. Rawshani, S. Franzén, N. Sattar, B. Eliasson, A.-M. Svensson, B. Zethelius, M. Miftaraj, D. K. McGuire, A. Rosengren, S. Gudbjörnsdottir, Risk Factors, Mortality, and Cardiovascular Outcomes in Patients with Type 2 Diabetes. N. Engl. J. Med. 379, 633-644 (2018).
7. H. Naci, M. Salcher-Konrad, S. Dias, M. R. Blum, S. A. Sahoo, D. Nunan, J. P. A. Ioannidis, How does exercise treatment compare with antihypertensive medications? A network meta-analysis of 391 randomised controlled trials assessing exercise and medication effects on systolic blood pressure. Br. J. Sports Med. 53 (2019), pp. 859-869.
8. W. C. Knowler, E. Barrett-Connor, S. E. Fowler, R. F. Hamman, J. M. Lachin, E. A. Walker, D. M. Nathan, D. P. P. R. Group, Reduction of the incidence of type 2 diabetes with lifestyle intervention or metformin. N. Engl. J. Med. 346, 393-403 (2002).
9. Diabetes Prevention Program Research Group, W. C. Knowler, S. E. Fowler, R. F. Hamman, C. A. Christophi, H. J. Hoffman, A. T. Brenneman, J. O. Brown-Friday, R. Goldberg, E. Venditti, D. M. Nathan, 10-year follow-up of diabetes incidence and weight loss in the Diabetes Prevention Program Outcomes Study. Lancet. 374, 1677-1686 (2009).
10. J. A. Sanford, C. D. Nogiec, M. E. Lindholm, J. N. Adkins, D. Amar, S. Dasari, J. K. Drugan, F. M. Fernandez, S. Radom-Aizik, S. Schenk, M. P. Snyder, R. P. Tracy, P. Vanderboom, S. Trappe, M. J. Walsh, T. M. T. of P. A. Consortium, Molecular Transducers of Physical Activity Consortium (MoTrPAC): Mapping the Dynamic Responses to Exercise. Cell. 181, 1464-1474 (2020).
11. L. D. Roberts, P. Boström, J. F. O'Sullivan, R. T. Schinzel, G. D. Lewis, A. Dejam, Y.-K. Lee, M. J. Palma, S. Calhoun, A. Georgiadi, M.-H. Chen, V. S. Ramachandran, M. G. Larson, C. Bouchard, T. Rankinen, A. L. Souza, C. B. Clish, T. J. Wang, J. L. Estall, A. A. Soukas, C. A. Cowan, B. M. Spiegelman, R. E. Gerszten, β-Aminoisobutyric acid induces browning of white fat and hepatic β-oxidation and is inversely correlated with cardiometabolic risk factors. Cell Metab. 19, 96-108 (2014).
12. K. I. Stanford, M. D. Lynes, H. Takahashi, L. A. Baer, P. J. Arts, F. J. May, A. C. Lehnig, R. J. W. Middelbeek, J. J. Richard, K. So, E. Y. Chen, F. Gao, N. R. Narain, G. Distefano, V. K. Shettigar, M. F. Hirshman, M. T. Ziolo, M. A. Kiebish, Y. H. Tseng, P. M. Coen, L. J. Goodyear, 12,13-diHOME: An Exercise-Induced Lipokine that Increases Skeletal Muscle Fatty Acid Uptake. Cell Metab. 27, 1111-1120.e3 (2018).
13. A. Reddy, L. H. M. Bozi, O. K. Yaghi, E. L. Mills, H. Xiao, H. E. Nicholson, M. Paschini, J. A. Paulo, R. Garrity, D. Laznik-Bogoslavski, J. C. B. Ferreira, C. S. Carl, K. A. Sjøberg, J. F. P. Wojtaszewski, J. F. Jeppesen, B. Kiens, S. P. Gygi, E. A. Richter, D. Mathis, E. T. Chouchani, pH-Gated Succinate Secretion Regulates Muscle Remodeling in Response to Exercise. Cell. 183, 62-75.e17 (2020).
14. Y. Yuan, P. Xu, Q. Jiang, X. Cai, T. Wang, W. Peng, J. Sun, C. Zhu, C. Zhang, D. Yue, Z. He, J. Yang, Y. Zeng, M. Du, F. Zhang, L. Ibrahimi, S. Schaul, Y. Jiang, J. Wang, J. Sun, Q. Wang, L. Liu, S. Wang, L. Wang, X. Zhu, P. Gao, Q. Xi, C. Yin, F. Li, G. Xu, Y. Zhang, G. Shu, Exercise-induced α-ketoglutaric acid stimulates muscle hypertrophy and fat loss through OXGR1-dependent adrenal activation. EMBO J. 39, e103304 (2020).
15. R. S. Jansen, R. Addie, R. Merkx, A. Fish, S. Mahakena, O. B. Bleijerveld, M. Altelaar, L. IJlst, R. J. Wanders, P. Borst, K. Van De Wetering, N-lactoyl-amino acids are ubiquitous metabolites that originate from CNDP2-mediated reverse proteolysis of lactate and amino acids. Proc. Natl. Acad. Sci. U.S.A 112, 6601-6606 (2015).
16. C. Wu, C. Orozco, J. Boyer, M. Leglise, J. Goodale, S. Batalov, C. L. Hodge, J. Haase, J. Janes, J. W. Huss, A. I. Su, BioGPS: An extensible and customizable portal for querying and organizing gene annotation resources. Genome Biol. 10, R130 (2009).
17. The Tabula Muris Consortium, Single-cell transcriptomics of 20 mouse organs creates a Tabula Muris. Nature. 562, 367-372 (2018).
18. J.-W. Sohn, J. K. Elmquist, K. W. Williams, Neuronal circuits that regulate feeding behavior and metabolism. Trends Neurosci. 36, 504-512 (2013).
19. M. J. Krashes, S. Koda, C. P. Ye, S. C. Rogan, A. C. Adams, D. S. Cusher, E. Maratos-Flier, B. L. Roth, B. B. Lowell, Rapid, reversible activation of AgRP neurons drives feeding behavior in mice. J. Clin. Invest. 121, 1424-1428 (2011).
20. S. Luquet, F. A. Perez, T. S. Hnasko, R. D. Palmiter, NPY/AgRP neurons are essentials for feeding in adult mice but can be ablated in neonates. Science (80-.). 310, 683-685 (2005).
21. Y. Aponte, D. Atasoy, S. M. Sternson, AGRP neurons are sufficient to orchestrate feeding behavior rapidly and without training. Nat. Neurosci. 14, 351-355 (2011).
22. G. J. Morton, D. E. Cummings, D. G. Baskin, G. S. Barsh, M. W. Schwartz, Central nervous system control of food intake and body weight. Nature. 443, 289-295 (2006).
23. Y. Xu, J. K. Elmquist, M. Fukuda, Central nervous control of energy and glucose balance: Focus on the central melanocortin system. Ann. N. Y. Acad. Sci. 1243, 1-14 (2011).
24. C. Zhan, J. Zhou, Q. Feng, J. en Zhang, S. Lin, J. Bao, P. Wu, M. Luo, Acute and long-term suppression of feeding behavior by POMC neurons in the brainstem and hypothalamus, respectively. J. Neurosci. 33, 3624-3632 (2013).
25. K. Contrepois, S. Wu, K. J. Moneghetti, D. Homburg, S. Ahadi, M. S. Tsai, A. A. Metwally, E. Wei, B. Lee-McMullen, J. V. Quijada, S. Chen, J. W. Christle, M. Ellenberger, B. Balliu, S. Taylor, M. G. Durrant, D. A. Knowles, H. Choudhry, M. Ashland, A. Bahmani, B. Enslen, M. Amsallem, Y. Kobayashi, M. Avina, D. Perelman, S. M. Schüssler-Fiorenza Rose, W. Zhou, E. A. Ashley, S. B. Montgomery, H. Chaib, F. Haddad, M. P. Snyder, Molecular Choreography of Acute Exercise. Cell. 181, 1112-1130.e16 (2020).
26. B. K. Mani, C. M. Castorena, S. Osborne-Lawrence, P. Vijayaraghavan, N. P. Metzger, J. K. Elmquist, J. M. Zigman, Ghrelin mediates exercise endurance and the feeding response post-exercise. Mol. Metab. 9, 114-130 (2018).
27. M. C. Miletta, O. Iyilikci, M. Shanabrough, M. Sestan-Peša, A. Cammisa, C. J. Zeiss, M. O. Dietrich, T. L. Horvath, AgRP neurons control compulsive exercise and survival in an activity-based anorexia model. Nat. Metab. 2, 1204-1211 (2020).
28. Z. He, Y. Gao, A. L. Alhadeff, C. M. Castorena, Y. Huang, L. Lieu, S. Afrin, J. Sun, J. N. Betley, H. Guo, K. W. Williams, Cellular and synaptic reorganization of arcuate NPY/AgRP and POMC neurons after exercise. Mol. Metab. 18, 107-119 (2018).
29. K. Beaulieu, M. Hopkins, J. Blundell, G. Finlayson, Homeostatic and non-homeostatic appetite control along the spectrum of physical activity levels: An updated perspective. Physiol. Behav. 192, 23-29 (2018).
30. K. Deighton, R. Barry, C. E. Connon, D. J. Stensel, Appetite, gut hormone and energy intake responses to low volume sprint interval and traditional endurance exercise. Eur. J. Appl. Physiol. 113, 1147-1156 (2013).
31. J. Dorling, D. R. Broom, S. F. Burns, D. J. Clayton, K. Deighton, L. J. James, J. A. King, M. Miyashita, A. E. Thackray, R. L. Batterham, D. J. Stensel, Acute and chronic effects of exercise on appetite, energy intake, and appetite-related hormones: The modulating effect of adiposity, sex, and habitual physical activity. Nutrients. 10, 1140 (2018).
32. A. P. Coll, M. Chen, P. Taskar, D. Rimmington, S. Patel, J. A. Tadross, I. Cimino, M. Yang, P. Welsh, S. Virtue, D. A. Goldspink, E. L. Miedzybrodzka, A. R. Konopka, R. R. Esponda, J. T. J. Huang, Y. C. L. Tung, S. Rodriguez-Cuenca, R. A. Tomaz, H. P. Harding, A. Melvin, G. S. H. Yeo, D. Preiss, A. Vidal-Puig, L. Vallier, K. S. Nair, N. J. Wareham, D. Ron, F. M. Gribble, F. Reimann, N. Sattar, D. B. Savage, B. B. Allan, S. O'Rahilly, GDF15 mediates the effects of metformin on body weight and energy balance. Nature. 578, 444-448 (2020).
33. S. C. A. De Jager, B. Bermüdez, I. Bot, R. R. Koenen, M. Bot, A. Kavelaars, V. De Waard, C. J. Heijnen, F. J. G. Muriana, C. Weber, T. J. C. Van Berkel, J. Kuiper, S. J. Lee, R. Abia, E. A. L. Biessen, Growth differentiation factor 15 deficiency protects against atherosclerosis by attenuating CCR2-mediated macrophage chemotaxis. J. Exp. Med. 208, 217-225 (2011).
34. M. Kleinert, C. Clemmensen, K. A. Sjøberg, C. S. Carl, J. F. Jeppesen, J. F. P. Wojtaszewski, B. Kiens, E. A. Richter, Exercise increases circulating GDF15 in humans. Mol. Metab. 9, 187-191 (2018).
35. C. Martins, L. M. Morgan, S. R. Bloom, M. D. Robertson, Effects of exercise on gut peptides, energy intake and appetite. J. Endocrinol. 193, 251-258 (2007).
36. S. Vatansever-Ozen, G. Tiryaki-Sonmez, G. Bugdayci, G. Ozen, The effects of exercise on food intake and hunger: Relationship with acylated ghrelin and leptin. J. Sport. Sci. Med. 10, 283-291 (2011).
37. B. S. and B. K. P. Adam Steensberg, Gerrit van Hall, Takuya Osada, Massimo Sacchetti, Production of interleukin-6 in contracting human skeletal muscles can account for the exercise-induced increase in plasma interleukin-6. J. Physiol. 529, 237-242 (2000).
38. R. R. Rao, J. Z. Long, J. P. White, K. J. Svensson, J. Lou, I. Lokurkar, M. P. Jedrychowski, J. L. Ruas, C. D. Wrann, J. C. Lo, D. M. Camera, J. Lachey, S. Gygi, J. Seehra, J. A. Hawley, B. M. Spiegelman, Meteorin-like is a hormone that regulates immune-adipose interactions to increase beige fat thermogenesis. Cell. 157, 1279-1291 (2014).
39. P. Boström, J. Wu, M. P. Jedrychowski, A. Korde, L. Ye, J. C. Lo, K. A. Rasbach, E. A. Boström, J. H. Choi, J. Z. Long, S. Kajimura, M. C. Zingaretti, B. F. Vind, H. Tu, S. Cinti, K. Højlund, S. P. Gygi, B. M. Spiegelman, A PGC1-α-dependent myokine that drives brown-fat-like development of white fat and thermogenesis. 481, 463-468 (2012).
40. B. Ingerslev, J. S. Hansen, C. Hoffmann, J. O. Clemmesen, N. H. Secher, M. Scheler, M. Hrabě de Angelis, H. U. Haring, B. K. Pedersen, C. Weigert, P. Plomgaard, Angiopoietin-like protein 4 is an exercise-induced hepatokine in humans, regulated by glucagon and cAMP. Mol. Metab. 6, 1286-1295 (2017).
41. A. M. Horowitz, X. Fan, G. Bieri, L. K. Smith, C. I. Sanchez-Diaz, A. B. Schroer, G. Gontier, K. B. Casaletto, J. H. Kramer, K. E. Williams, S. A. Villeda, Blood factors transfer beneficial effects of exercise on neurogenesis and cognition to the aged brain. Science (80-.). 369, 167-173 (2020).
42. S. Norton, F. E. Matthews, D. E. Barnes, K. Yaffe, C. Brayne, Potential for primary prevention of Alzheimer's disease: An analysis of population-based data. Lancet Neurol. 13, 788-794 (2014).
43. M. Hamer, Y. Chida, Physical activity and risk of neurodegenerative disease: A systematic review of prospective evidence. Psychol. Med. 39, 3-11 (2009).

Notwithstanding the appended claims, the disclosure is also defined by the following clauses:

1. A method of treating a metabolic disorder in a subject, the method comprising administering an effective amount of an N-lactoyl-amino acid to the subject.

2. The method of Clause 1, wherein the N-lactoyl-amino acid is N-lactoyl-phenylalanine.

3. The method of any of Clauses 1-2, wherein the effective amount comprises an amount of the N-lactoyl-amino acid that when administered produces a plasma concentration of the N-lactoyl-amino acid comparable to that observed after physical activity.

4. The method of any of Clauses 1-3, wherein the effective amount ranges from 1 mg/kg to 500 mg/kg.

5. The method of any of Clauses 1-4, wherein the effective amount is administered once a day over a period of time ranging from 1 day to 60 days.

6. The method of any of Clauses 1-5, wherein the metabolic disorder is obesity.

7. The method of any of Clauses 1-6, wherein the metabolic disorder is an obesity-related metabolic disorder.

8. The method of any of Clauses 1-7, wherein the metabolic disorder is diabetes.

9. The method of any of Clauses 1-8, wherein the method reduces food intake by the subject compared to food intake by the subject before treatment.

10. The method of Clause 9, wherein cumulative food intake is reduced by 10% to 90%.

11. The method of any of Clauses 9-10, wherein average daily food intake is reduced by 10% to 90%.

12. The method of any of Clauses 1-11, wherein the method reduces a body weight of the subject compared to a body weight of the subject before treatment.

13. The method of Clause 12, wherein body weight is reduced by 1% to 50%.

14. The method of any of Clauses 1-13, wherein the method improves glucose homeostasis in the subject compared to glucose homeostasis in the subject before treatment.

15. The method of any of Clauses 1-14, wherein the method improves glucose clearance in the subject compared to glucose clearance in the subject before treatment.

16. The method of Clause 15, wherein glucose clearance is improved by 1% to 50%.

17. The method of any of Clauses 1-16, wherein the method reduces adipose tissue mass compared to adipose tissue mass in the subject before treatment.

18. The method of Clause 17, wherein the method reduces brown fat by 30% to 50%.

19. The method of any of Clauses 1-18, wherein the subject is an adult.

20. The method of any of Clauses 1-19, wherein the subject is a mammal.

21. The method of any of Clauses 1-20, wherein the subject is a human.

22. The method of any of Clauses 1-21, wherein the administering comprises oral administration.

23. The method of any of Clauses 1-22, wherein the administering comprises parenteral administration.

24. The method of any of Clauses 1-23, wherein the administering comprises intraperitoneal administration.

25. The method of any of Clauses 1-24, wherein the method comprises administering the N-lactoyl-amino acid in combination with one or more therapies for treating the metabolic disorder.

26. The method of Clause 25, wherein the one or more therapies comprises an active agent for treating the metabolic disorder.

27. The method of any of Clauses 25-26, wherein the one or more therapies comprises physical activity.

28. The method of any of Clauses 25-27, wherein the one or more therapies comprises a low-calorie diet.

29. The method of any of Clauses 25-28, wherein the one or more therapies comprises surgical intervention.

30. The method of any of Clauses 25-29, wherein the one or more therapies comprises use of a weight loss device.

31. A pharmaceutical formulation comprising:
an amount of an N-lactoyl-amino acid effective to treat a metabolic disorder; and
an excipient.

32. The pharmaceutical formulation of Clause 31, wherein the N-lactoyl-amino acid is N-lactoyl-phenylalanine.

33. The pharmaceutical formulation of any of Clauses 31-32, wherein the effective amount ranges from 1 mg/kg to 500 mg/kg.

34. The pharmaceutical formulation of any of Clauses 31-33, wherein the pharmaceutical formulation is a capsule or tablet.

35. The pharmaceutical formulation of any of Clauses 31-33, wherein the pharmaceutic formulation is a parenteral formulation.

36. The pharmaceutical formulation of any of Clauses 31-33, wherein the pharmaceutical formulation is an intraperitoneal formulation.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A method of treating a metabolic disorder in a subject, the method comprising administering an effective amount of an N-lactoyl-amino acid to the subject.

2. The method of claim 1, wherein the N-lactoyl-amino acid is N-lactoyl-phenylalanine.

3. The method of claim 1, wherein the effective amount comprises an amount of the N-lactoyl-amino acid that when administered produces a plasma concentration of the N-lactoyl-amino acid comparable to that observed after physical activity.

4. The method of claim 1, wherein the metabolic disorder is obesity.

5. The method of claim 1, wherein the metabolic disorder is an obesity-related metabolic disorder.

6. The method of claim 1, wherein the metabolic disorder is diabetes.

7. The method of claim 1, wherein the method reduces food intake by the subject compared to food intake by the subject before treatment.

8. The method of claim 1, wherein the method reduces a body weight of the subject compared to a body weight of the subject before treatment.

9. The method of claim 1, wherein the method improves glucose homeostasis in the subject compared to glucose homeostasis and/or glucose clearance in the subject before treatment.

10. The method of claim 1, wherein the method reduces adipose tissue mass compared to adipose tissue mass in the subject before treatment.

11. The method of claim 1, wherein the method comprises administering the N-lactoyl-amino acid in combination with one or more therapies for treating the metabolic disorder.

12. The method of claim 11, wherein the one or more therapies comprises an active agent for treating the metabolic disorder.

13. The method of claim 11, wherein the one or more therapies comprises a low-calorie diet.

14. The method of claim 11, wherein the one or more therapies comprises surgical intervention.

15. A pharmaceutical formulation comprising:
an amount of an N-lactoyl-amino acid effective to treat a metabolic disorder; and
an excipient.

16. The pharmaceutical formulation of claim 15, wherein the N-lactoyl-amino acid is N-lactoyl-phenylalanine.

17. The pharmaceutical formulation of claim 15, wherein the effective amount ranges from 1 mg/kg to 500 mg/kg.

18. The pharmaceutical formulation of claim 15, wherein the pharmaceutical formulation is a capsule or tablet.

19. The pharmaceutical formulation of claim 15, wherein the pharmaceutic formulation is a parenteral formulation.

20. The pharmaceutical formulation of claim 15, wherein the pharmaceutical formulation is an intraperitoneal formulation.


SEQUENCE LISTING (7 sequences)

SEQ ID NO: 1 - DNA, 20 bp, Artificial sequence
  synthetic sequence
cagtgaaatgagatccgtca

SEQ ID NO: 2 - DNA, 25 bp, Artificial sequence
  synthetic sequence
caccgcagtgaaatgagatccgtca

SEQ ID NO: 3 - DNA, 24 bp, Artificial sequence
  synthetic sequence
aaactgacggatctcatttcactg

SEQ ID NO: 4 - DNA, 21 bp, Artificial sequence
  synthetic sequence
cagatggctcggagataccac

SEQ ID NO: 5 - DNA, 22 bp, Artificial sequence
  synthetic sequence
ttcccgctccaccaaggtgaag

SEQ ID NO: 6 - DNA, 25 bp, Artificial sequence
  synthetic sequence
gctctgtaagggaaagagatgaccc

SEQ ID NO: 7 - DNA, 26 bp, Artificial sequence
  synthetic sequence
aataggacatacccagttctgtgagg